SEQUENCE LISTING (8 sequences)

SEQ ID NO: 1 - DNA, 1962 bp, synthetic construct
  source 1..1962: mol_type = other DNA
atgcgacctctgaggccacgggctgcgttgttggctcttttggcgtccctgttggccgcaccgcctgtcgctccagcagaagcaccccacctcgtgcatgttgatgcagcacgagcactgtggccgcttcggaggttttggaggtcaacggggttctgtcctccattgccacactctcaagcggatcagtatgtgttgtcttgggatcagcaactgaacctggcctatgtcggtgccgtacctcatcgcggaattaaacaggttcgaacccattggctccttgagttggttacgacacggggcagcacaggaaggggactctcttacaatttcactcacttggacggataccttgacctcctgcgagagaatcagctgcttcccggtttcgagttgatggggtcagctagcggacacttcactgatttcgaggataagcaacaagtttttgagtggaaagacctcgtgtcatcacttgcccggaggtatatagggcgctacgggcttgcgcacgtaagtaagtggaactttgagacgtggaatgagcctgatcaccacgattttgacaatgtttctatgacgatgcagggctttctcaactactatgacgcgtgttctgaaggactgagagcagccagtccggcactccgcctggggggcccaggggactccttccacacaccgcctcggtccccgctttcttgggggctgttgcgacactgtcatgacggtacgaacttttttacaggagaagcgggcgtccgcctcgattatatttctctgcatcgaaaaggtgcgaggtcctcaatctctatcctggagcaggagaaggtcgtggcacaacaaattagacaactctttcctaaatttgccgacacccctatctataatgatgaggccgaccctcttgtgggttggtcacttccgcaaccctggcgggcggatgtgacatatgcagcaatggtagtcaaagtgatcgcgcaacatcaaaacttgctcctggcaaacacgacatccgcttttccgtatgccttgctttcaaacgataacgctttcctttcataccacccgcatccctttgcacagcgaaccctcacagcgcggttccaggtcaataacacacgacctcctcacgtccagttgttgagaaaacctgtgcttactgccatgggacttctggcacttctcgacgaggaacagctgtgggcggaggtatctcaagcagggactgtattggatagcaaccacaccgtaggggtcctcgcctccgcgcacaggccacaaggacctgctgatgcttggcgagctgcagttctgatctacgccagtgacgacactagggcccatccgaacagatcagtcgcggtgacgctgaggctccgcggagtgccccctggtcccggtctcgtttacgtcactcgctacctcgacaacggactctgttctccggatggcgagtggagaagacttggacggcctgttttcccgaccgcggaacaattccggcgaatgagggccgctgaggacccagtagcggcagcgccaagacctctccccgctgggggccgcctcacccttagacccgctctcaggctccccagtcttctcttggtacacgtatgtgcccgacctgagaaaccccctggccaggtcactcgcttgcgagctcttcctttgactcaaggacaacttgtcctcgtttggagcgacgagcatgtgggctctaaatgcctctggacttatgaaatacagttctcacaagatggcaaagcatacaccccggtatctaggaaaccctcaacgttcaacctgtttgtattctcccccgacacgggagccgtgtcaggctcctaccgagttcgggccttggactactgggctaggcccggccccttctccgatccagtcccatatttggaggtccctgttccgcgcggaccccccagcccgggcaatccctga

SEQ ID NO: 2 - DNA, 1947 bp, synthetic construct
  source 1..1947: mol_type = other DNA
atgcgtcccctgcgcccccgcgccgcgctgctggcgctcctggcctcgctcctggccgcgcccccggtggccccggccgaggccccgcacctggtgcatgtggacgcggcccgcgcgctgtggcccctgcggcgcttctggaggagcacaggcttctgccccccgctgccacacagccaggctgaccagtacgtcctcagctgggaccagcagctcaacctcgcctatgtgggcgccgtccctcaccgcggcatcaagcaggtccggacccactggctgctggagcttgtcaccaccagggggtccactggacggggcctgagctacaacttcacccacctggacgggtacctggaccttctcagggagaaccagctcctcccagggtttgagctgatgggcagcgcctcgggccacttcactgactttgaggacaagcagcaggtgtttgagtggaaggacttggtctccagcctggccaggagatacatcggtaggtacggactggcgcatgtttccaagtggaacttcgagacgtggaatgagccagaccaccacgactttgacaacgtctccatgaccatgcaaggcttcctgaactactacgatgcctgctcggagggtctgcgcgccgccagccccgccctgcggctgggaggccccggcgactccttccacaccccaccgcgatccccgctgagctggggcctcctgcgccactgccacgacggtaccaacttcttcactggggaggcgggcgtgcggctggactacatctccctccacaggaagggtgcgcgcagctccatctccatcctggagcaggagaaggtcgtcgcgcagcagatccggcagctcttccccaagttcgcggacacccccatttacaacgacgaggcggacccgctggtgggctggtccctgccacagccgtggagggcggacgtgacctacgcggccatggtggtgaaggtcatcgcgcagcatcagaacctgctactggccaacaccacctccgccttcccctacgcgctcctgagcaacgacaatgccttcctgagctaccacccgcaccccttcgcgcagcgcacgctcaccgcgcgcttccaggtcaacaacacccgcccgccgcacgtgcagctgttgcgcaagccggtgctcacggccatggggctgctggcgctgctggatgaggagcagctctgggccgaagtgtcgcaggccgggaccgtcctggacagcaaccacacggtgggcgtcctggccagcgcccaccgcccccagggcccggccgacgcctggcgcgccgcggtgctgatctacgcgagcgacgacacccgcgcccaccccaaccgcagcgtcgcggtgaccctgcggctgcgcggggtgccccccggcccgggcctggtctacgtcacgcgctacctggacaacgggctctgcagccccgacggcgagtggcggcgcctgggccggcccgtcttccccacggcagagcagttccggcgcatgcgcgcggctgaggacccggtggccgcggcgccccgccccttacccgccggcggccgcctgaccctgcgccccgcgctgcggctgccgtcgcttttgctggtgcacgtgtgtgcgcgccccgagaagccgcccgggcaggtcacgcggctccgcgccctgcccctgacccaagggcagctggttctggtctggtcggatgaacacgtgggctccaagtgcctgtggacatacgagatccagttctctcaggacggtaaggcgtacaccccggtcagcaggaagccatcgaccttcaacctctttgtgttcagcccagacacaggtgctgtctctggctcctaccgagttcgagccctggactactgggcccgaccaggccccttctcggaccctgtgccgtacctggaggtccctgtgtccccgggcaatccatga

SEQ ID NO: 3 - DNA, 1947 bp, synthetic construct
  source 1..1947: mol_type = other DNA
atgcgacctctgaggccacgggctgcgttgttggctcttttggcgtccctgttggccgcaccgcctgtcgctccagcagaagcaccccacctcgtgcatgttgatgcagcacgagcactgtggccgcttcggaggttttggaggtcaacggggttctgtcctccattgccacactctcaagcggatcagtatgtgttgtcttgggatcagcaactgaacctggcctatgtcggtgccgtacctcatcgcggaattaaacaggttcgaacccattggctccttgagttggttacgacacggggcagcacaggaaggggactctcttacaatttcactcacttggacggataccttgacctcctgcgagagaatcagctgcttcccggtttcgagttgatggggtcagctagcggacacttcactgatttcgaggataagcaacaagtttttgagtggaaagacctcgtgtcatcacttgcccggaggtatatagggcgctacgggcttgcgcacgtaagtaagtggaactttgagacgtggaatgagcctgatcaccacgattttgacaatgtttctatgacgatgcagggctttctcaactactatgacgcgtgttctgaaggactgagagcagccagtccggcactccgcctggggggcccaggggactccttccacacaccgcctcggtccccgctttcttgggggctgttgcgacactgtcatgacggtacgaacttttttacaggagaagcgggcgtccgcctcgattatatttctctgcatcgaaaaggtgcgaggtcctcaatctctatcctggagcaggagaaggtcgtggcacaacaaattagacaactctttcctaaatttgccgacacccctatctataatgatgaggccgaccctcttgtgggttggtcacttccgcaaccctggcgggcggatgtgacatatgcagcaatggtagtcaaagtgatcgcgcaacatcaaaacttgctcctggcaaacacgacatccgcttttccgtatgccttgctttcaaacgataacgctttcctttcataccacccgcatccctttgcacagcgaaccctcacagcgcggttccaggtcaataacacacgacctcctcacgtccagttgttgagaaaacctgtgcttactgccatgggacttctggcacttctcgacgaggaacagctgtgggcggaggtatctcaagcagggactgtattggatagcaaccacaccgtaggggtcctcgcctccgcgcacaggccacaaggacctgctgatgcttggcgagctgcagttctgatctacgccagtgacgacactagggcccatccgaacagatcagtcgcggtgacgctgaggctccgcggagtgccccctggtcccggtctcgtttacgtcactcgctacctcgacaacggactctgttctccggatggcgagtggagaagacttggacggcctgttttcccgaccgcggaacaattccggcgaatgagggccgctgaggacccagtagcggcagcgccaagacctctccccgctgggggccgcctcacccttagacccgctctcaggctccccagtcttctcttggtacacgtatgtgcccgacctgagaaaccccctggccaggtcactcgcttgcgagctcttcctttgactcaaggacaacttgtcctcgtttggagcgacgagcatgtgggctctaaatgcctctggacttatgaaatacagttctcacaagatggcaaagcatacaccccggtatctaggaaaccctcaacgttcaacctgtttgtattctcccccgacacgggagccgtgtcaggctcctaccgagttcgggccttggactactgggctaggcccggccccttctccgatccagtcccatatttggaggtccctgttagcccgggcaatccctga

SEQ ID NO: 4 - DNA, 6059 bp, synthetic construct
  source 1..6059: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggggttatttcgtattttatttagatctgatggtcagggattgcccgggctaacagggacctccaaatatgggactggatcggagaaggggccgggcctagcccagtagtccaaggcccgaactcggtaggagcctgacacggctcccgtgtcgggggagaatacaaacaggttgaacgttgagggtttcctagataccggggtgtatgctttgccatcttgtgagaactgtatttcataagtccagaggcatttagagcccacatgctcgtcgctccaaacgaggacaagttgtccttgagtcaaaggaagagctcgcaagcgagtgacctggccagggggtttctcaggtcgggcacatacgtgtaccaagagaagactggggagcctgagagcgggtctaagggtgaggcggcccccagcggggagaggtcttggcgctgccgctactgggtcctcagcggccctcattcgccggaattgttccgcggtcgggaaaacaggccgtccaagtcttctccactcgccatccggagaacagagtccgttgtcgaggtagcgagtgacgtaaacgagaccgggaccagggggcactccgcggagcctcagcgtcaccgcgactgatctgttcggatgggccctagtgtcgtcactggcgtagatcagaactgcagctcgccaagcatcagcaggtccttgtggcctgtgcgcggaggcgaggacccctacggtgtggttgctatccaatacagtccctgcttgagatacctccgcccacagctgttcctcgtcgagaagtgccagaagtcccatggcagtaagcacaggttttctcaacaactggacgtgaggaggtcgtgtgttattgacctggaaccgcgctgtgagggttcgctgtgcaaagggatgcgggtggtatgaaaggaaagcgttatcgtttgaaagcaaggcatacggaaaagcggatgtcgtgtttgccaggagcaagttttgatgttgcgcgatcactttgactaccattgctgcatatgtcacatccgcccgccagggttgcggaagtgaccaacccacaagagggtcggcctcatcattatagataggggtgtcggcaaatttaggaaagagttgtctaatttgttgtgccacgaccttctcctgctccaggatagagattgaggacctcgcaccttttcgatgcagagaaatataatcgaggcggacgcccgcttctcctgtaaaaaagttcgtaccgtcatgacagtgtcgcaacagcccccaagaaagcggggaccgaggcggtgtgtggaaggagtcccctgggccccccaggcggagtgccggactggctgctctcagtccttcagaacacgcgtcatagtagttgagaaagccctgcatcgtcatagaaacattgtcaaaatcgtggtgatcaggctcattccacgtctcaaagttccacttacttacgtgcgcaagcccgtagcgccctatatacctccgggcaagtgatgacacgaggtctttccactcaaaaacttgttgcttatcctcgaaatcagtgaagtgtccgctagctgaccccatcaactcgaaaccgggaagcagctgattctctcgcaggaggtcaaggtatccgtccaagtgagtgaaattgtaagagagtccccttcctgtgctgccccgtgtcgtaaccaactcaaggagccaatgggttcgaacctgtttaattccgcgatgaggtacggcaccgacataggccaggttcagttgctgatcccaagacaacacatactgatccgcttgagagtgtggcaatggaggacagaaccccgttgacctccaaaacctccgaagcggccacagtgctcgtgctgcatcaacatgcacgaggtggggtgcttctgctggagcgacaggcggtgcggccaacagggacgccaaaagagccaacaacgcagcccgtggcctcagaggtcgcatggcgaccggtcgagcggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtccgggggtcgttgggcggtcagccaggcgggccatttaccgtaagttatgtaacgaaagcttcctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 5 - DNA, 6074 bp, synthetic construct
  source 1..6074: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggggttatttcgtattttatttagatctgatggtcagggattgcccgggctggggggtccgcgcggaacagggacctccaaatatgggactggatcggagaaggggccgggcctagcccagtagtccaaggcccgaactcggtaggagcctgacacggctcccgtgtcgggggagaatacaaacaggttgaacgttgagggtttcctagataccggggtgtatgctttgccatcttgtgagaactgtatttcataagtccagaggcatttagagcccacatgctcgtcgctccaaacgaggacaagttgtccttgagtcaaaggaagagctcgcaagcgagtgacctggccagggggtttctcaggtcgggcacatacgtgtaccaagagaagactggggagcctgagagcgggtctaagggtgaggcggcccccagcggggagaggtcttggcgctgccgctactgggtcctcagcggccctcattcgccggaattgttccgcggtcgggaaaacaggccgtccaagtcttctccactcgccatccggagaacagagtccgttgtcgaggtagcgagtgacgtaaacgagaccgggaccagggggcactccgcggagcctcagcgtcaccgcgactgatctgttcggatgggccctagtgtcgtcactggcgtagatcagaactgcagctcgccaagcatcagcaggtccttgtggcctgtgcgcggaggcgaggacccctacggtgtggttgctatccaatacagtccctgcttgagatacctccgcccacagctgttcctcgtcgagaagtgccagaagtcccatggcagtaagcacaggttttctcaacaactggacgtgaggaggtcgtgtgttattgacctggaaccgcgctgtgagggttcgctgtgcaaagggatgcgggtggtatgaaaggaaagcgttatcgtttgaaagcaaggcatacggaaaagcggatgtcgtgtttgccaggagcaagttttgatgttgcgcgatcactttgactaccattgctgcatatgtcacatccgcccgccagggttgcggaagtgaccaacccacaagagggtcggcctcatcattatagataggggtgtcggcaaatttaggaaagagttgtctaatttgttgtgccacgaccttctcctgctccaggatagagattgaggacctcgcaccttttcgatgcagagaaatataatcgaggcggacgcccgcttctcctgtaaaaaagttcgtaccgtcatgacagtgtcgcaacagcccccaagaaagcggggaccgaggcggtgtgtggaaggagtcccctgggccccccaggcggagtgccggactggctgctctcagtccttcagaacacgcgtcatagtagttgagaaagccctgcatcgtcatagaaacattgtcaaaatcgtggtgatcaggctcattccacgtctcaaagttccacttacttacgtgcgcaagcccgtagcgccctatatacctccgggcaagtgatgacacgaggtctttccactcaaaaacttgttgcttatcctcgaaatcagtgaagtgtccgctagctgaccccatcaactcgaaaccgggaagcagctgattctctcgcaggaggtcaaggtatccgtccaagtgagtgaaattgtaagagagtccccttcctgtgctgccccgtgtcgtaaccaactcaaggagccaatgggttcgaacctgtttaattccgcgatgaggtacggcaccgacataggccaggttcagttgctgatcccaagacaacacatactgatccgcttgagagtgtggcaatggaggacagaaccccgttgacctccaaaacctccgaagcggccacagtgctcgtgctgcatcaacatgcacgaggtggggtgcttctgctggagcgacaggcggtgcggccaacagggacgccaaaagagccaacaacgcagcccgtggcctcagaggtcgcatggcgaccggtcgagcggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtccgggggtcgttgggcggtcagccaggcgggccatttaccgtaagttatgtaacgaaagcttcctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 6 - DNA, 6074 bp, synthetic construct
  source 1..6074: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggggttatttcgtattttatttagatctgatggtcatggattgcccggggatgggggccctcttggcacagggacctccaggtacggcacagggtccgagaaggggcctggtcgggcccagtagtccagggctcgaactcggtaggagccagagacagcacctgtgtctgggctgaacacaaagaggttgaaggtcgatggcttcctgctgaccggggtgtacgccttaccgtcctgagagaactggatctcgtatgtccacaggcacttggagcccacgtgttcatccgaccagaccagaaccagctgcccttgggtcaggggcagggcgcggagccgcgtgacctgcccgggcggcttctcggggcgcgcacacacgtgcaccagcaaaagcgacggcagccgcagcgcggggcgcagggtcaggcggccgccggcgggtaaggggcggggcgccgcggccaccgggtcctcagccgcgcgcatgcgccggaactgctctgccgtggggaagacgggccggcccaggcgccgccactcgccgtcggggctgcagagcccgttgtccaggtagcgcgtgacgtagaccaggcccgggccggggggcaccccgcgcagccgcagggtcaccgcgacgctgcggttggggtgggcgcgggtgtcgtcgctcgcgtagatcagcaccgcggcgcgccaggcgtcggccgggccctgggggcggtgggcgctggccaggacgcccaccgtgtggttgctgtccaggacggtcccggcctgcgacacttcggcccagagctgctcctcatccagcagcgccagcagccccatggccgtgagcaccggcttgcgcaacagctgcacgtgcggcgggcgggtgttgttgacctggaagcgcgcggtgagcgtgcgctgcgcgaaggggtgcgggtggtagctcaggaaggcattgtcgttgctcaggagcgcgtaggggaaggcggaggtggtgttggccagtagcaggttctgatgctgcgcgatgaccttcaccaccatggccgcgtaggtcacgtccgccctccacggctgtggcagggaccagcccaccagcgggtccgcctcgtcgttgtaaatgggggtgtccgcgaacttggggaagagctgccggatctgctgcgcgacgaccttctcctgctccaggatggagatggagctgcgcgcacccttcctgtggagggagatgtagtccagccgcacgcccgcctccccagtgaagaagttggtaccgtcgtggcagtggcgcaggaggccccagctcagcggggatcgcggtggggtgtggaaggagtcgccggggcctcccagccgcagggcggggctggcggcgcgcagaccctccgagcaggcatcgtagtagttcaggaagccttgcatggtcatggagacgttgtcaaagtcgtggtggtctggctcattccacgtctcgaagttccacttggaaacatgcgccagtccgtacctaccgatgtatctcctggccaggctggagaccaagtccttccactcaaacacctgctgcttgtcctcaaagtcagtgaagtggcccgaggcgctgcccatcagctcaaaccctgggaggagctggttctccctgagaaggtccaggtacccgtccaggtgggtgaagttgtagctcaggccccgtccagtggaccccctggtggtgacaagctccagcagccagtgggtccggacctgcttgatgccgcggtgagggacggcgcccacataggcgaggttgagctgctggtcccagctgaggacgtactggtcagcctggctgtgtggcagcggggggcagaagcctgtgctcctccagaagcgccgcaggggccacagcgcgcgggccgcgtccacatgcaccaggtgcggggcctcggccggggccaccgggggcgcggccaggagcgaggccaggagcgccagcagcgcggcgcgggggcgcaggggacgcatggcgaccggtcgagcggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtccgggggtcgttgggcggtcagccaggcgggccatttaccgtaagttatgtaacgaaagcttcctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 7 - DNA, 228 bp, synthetic construct
  source 1..228: mol_type = other DNA
cggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtccgggggtcgttgggcggtcagccaggcgggccatttaccgtaagttatgtaacga

SEQ ID NO: 8 - DNA, 16 bp, synthetic construct
  source 1..16: mol_type = other DNA
aaataaaatacgaaat

(19) United States
(12) Patent Application Publication
(10) Pub. No.: US 2026/0132393 A1
(43) Pub. Date: May 14, 2026

(54) AAV-IDUA VECTOR FOR TREATMENT OF MPS I

(71) Applicant: The University of North Carolina at Chapel Hill, Chapel Hill, NC (US)

(72) Inventors: Haiyan Fu, Durham, NC (US); Tierra Bobo, Mebane, NC (US); Hyoukbum Kwon, Chapel Hill, NC (US)

(21) Appl. No.: 18/704,645

(22) PCT Filed: Oct. 27, 2022
(86) PCT No.: PCT/US2022/078746

§ 371 Date: Apr. 25, 2024

Related U.S. Application Data
(60) Provisional application No. 63/272,501, filed on Oct. 27, 2021.

(51) Int. Cl. C12N 9/24; A61K 38/47; A61K 48/00; A61P 5/00; C12N 15/86

(52) U.S. Cl. CPC C12N 9/2402; A61K 38/47; A61K 48/005; A61P 5/00; C12N 15/86; C12Y 302/01076; C12N 2750/14143; C12N 2750/14152; C12N 2830/50

(57) ABSTRACT

This invention relates to viral vectors for delivery of alpha-L-iduronidase to a subject and methods of using the same for treatment and prevention of mucopolysaccharidosis I in a subject, including codon optimized and/or truncated human IDUA nucleotide sequences. Moreover, the invention relates to an AAV vector genome comprising the nucleic acid of the invention, an AAV particle comprising the AAV vector genome, and a pharmaceutical composition comprising the AAV particle.

### STATEMENT OF PRIORITY

This application claims the benefit of U.S. Provisional Application Ser. No. 63/272,501, filed Oct. 27, 2021, the entire contents of which are incorporated by reference herein.

### FIELD OF THE INVENTION

This invention relates to viral vectors for delivery of alpha-L-iduronidase to a subject and methods of using the same for treatment of mucopolysaccharidosis I in a subject.

### BACKGROUND OF THE INVENTION

Mucopolysaccharidosis I (MPS I) is an autosomal recessive lysosomal storage disorder (LSD) caused by a defect in α-L-iduronidase (IDUA), a lysosomal enzyme essential for the stepwise degradation of biologically important glycosaminoglycans (GAGs), heparan sulfates (HS) and dermatan sulfates (DS). The lack of or reduced IDUA activity results in the accumulation of undegraded or partially degraded HS and DS GAGs in cells in virtually all organs, leading to progressive multisystem disorders. Infants with MPS I appear normal at birth, but deteriorate rapidly between the ages of 4 months and 2 years. While the severity of the disease varies widely among individuals, clinically, there are 2 forms of MPS I, attenuated (Scheie syndrome) and severe (Hurler syndrome). While MPS I presents a spectrum of clinical phenotypes with heterogeneous mutations, all forms of the disease are biochemically indistinguishable, with progressive multisystem disorders. The severe form of MPS I (MPS IH) represents up to 80% of known cases. Children with MPS IH usually die of neurological deterioration and cardiorespiratory failure before 10 years of age, though some with attenuated MPS I (MPS IS) can live to adulthood.

Current MPS I treatments include IDUA enzyme replacement therapy (ERT) (ALDURAZYMER) delivered intravenously (IV) weekly, with demonstrated improvements in somatic symptoms in both severe and attenuated patients. A more promising treatment relies on allogeneic hematopoietic stem cell transplantation (HSCT) which has been used for the past 2-3 decades in MPS I patients. HSCT also benefits the somatic disorders of MPS I, with neurological impacts if performed early (<age 2 years). Otherwise, in general, no treatment is currently available to treat the CNS disorders in MPS IH.

Gene therapy targeting the lack of or reduced IDUA activity with broad delivery is ideal for treating MPS I if delivered to CNS and peripheral tissues, because of the potential for long-term endogenous production of recombinant enzymes and the by-stander effect of IDUA. The endogenous mannose-6-phosphate-mediated lysosomal trafficking pathway allows the secretion of 5-20% IDUA (and other lysosomal hydrolases), leading to a therapeutic by-stander effect/cross-correction on neighboring cells. Only a very low level of enzyme activity (1-10%) is needed to normalize metabolic function. Therefore, near optimal therapeutic benefit can be achieved by gene transfer to a minority of CNS cells as long as the distribution is homogeneous, without the need to treat every cell. Among the gene therapy strategies, recombinant adeno-associated virus (rAAV) is a potential vector for this application because the target tissue is largely post-mitotic. The demonstrated trans-blood brain barrier (BBB)-neurotropic AAV9 has offered a gene delivery tool for treating monogenic neurological diseases, targeting the root cause of these diseases. To date, systemic rAAV9) gene replacement and their associated gene therapy products have been demonstrated to be functionally beneficial with the potential for cure in animal models with neuropathic LSDs.

AAV normally packages as a single-stranded (ss) DNA genome that must be converted to double-stranded (ds) DNA after infection. Constructs less than half of the normal wild-type (WT) AAV genome size can be packaged as dimeric inverted repeat DNA molecules. These self-complementary (sc) genomes fold into ds DNA as soon as they are released from the capsid, bypassing the requirement for DNA synthesis by the host cells to convert the ss vector genome (vg) into active double-stranded DNA and providing faster and more efficient transduction.

There is a need in the art for improved viral vectors for expressing IDUA and treating MPS I.

### SUMMARY OF THE INVENTION

This invention is based on the finding that the use of self-complementary AAV vectors for delivery of IDUA to subjects with MPS I is effective to express IDUA, thereby treating MPS I. The invention further relates to a highly effective codon-optimized nucleotide sequence for IDUA. Thus, one aspect of the invention relates to a recombinant nucleic acid comprising a nucleotide sequence encoding human IDUA, wherein the nucleotide sequence has been codon-optimized for expression in human cells.

A further aspect of the invention relates to the use of a 228 bp truncated miniature cytomegalovirus promoter (mCMV), a 16 bp soluble neuropilin-1 (sNRP-1) Poly A signal, and miniaturized hIDUA^{op} cDNA (ΔhIDUA^{op}) or non-optimized, miniaturized hIDUA cDNA (ΔhIDUA), which enables the successful packaging of scAAV-ΔhIDUA^{op} or scAAV-ΔhIDUA, respectively.

Another aspect of the invention relates to an AAV vector genome comprising the nucleic acid of the invention, an AAV particle comprising the AAV vector genome, and a pharmaceutical composition comprising the AAV particle.

A further aspect of the invention relates to a method of producing a recombinant AAV particle, the method comprising: providing a cell in vitro with AAV Cap and AAV Rep coding sequences, the AAV vector genome of the invention, and helper functions for generating a productive AAV infection; and allowing assembly of the recombinant AAV particle and encapsidating the AAV vector genome.

An additional aspect of the invention relates to a method of delivering IDUA to a subject, comprising administering to the subject an effective amount of an AAV particle that expresses IDUA, thereby delivering IDUA to the subject.

Another aspect of the invention relates to a method of treating or delaying the onset of MPS I in a subject in need thereof, comprising administering to the subject a therapeutically effective amount of an AAV particle that expresses IDUA, thereby treating or delaying the onset of MPS I—in the subject.

These and other aspects of the invention are set forth in more detail in the description of the invention below.

### BRIEF DESCRIPTION OF THE DRAWINGS

FIG. 1 is a schematic structure of the rAAV9-mCMV-hIDUA viral vector genome. ITR: wt AAV2 terminal repeat; dITR: AAV2 terminal repeat with deletion of terminal resolution site to force generation of self-complementary dimeric genomes; hIDUA: human Iduronidase cDNA (1,962 bp); hIDUA^{op}: codon-optimized hIDUA cDNA (1,962 bp); ΔhIDUA^{op}: miniaturized hIDUA^{op} cDNA (1,947 bp); mCMV: truncated miniaturized human CMV promoter (228 bp); PA: sNRP-1 polyadenylation signals (16 bp).

FIGS. 2A-2B are gels showing the generation of scAAV9-mCMV-hIDUA vectors. HEK293 cells were co-transfected by ptrs-mCMV-hIDUA, ptrs-mCMV-hIDUA^{op}, or ptrs-mCMV-ΔhIDUA^{op}, and the helper plasmids pHELP and pAAV2/9. Purified AAV9 vector products were analyzed by alkaline denaturing gel electrophoresis. sc Ctr: a known scAAV9 vector; hIDUA: AAV9-mCMV-hIDUA; hIDUA^{op}: AAV9-mCMV-hIDUA^{op}; ΔhIDUA^{op}: AAV9-mCMV-ΔhIDUA^{op}; sc: scAAV vector genome; ss: single-stranded control vector genome.

FIGS. 3A-3C show AAV-hIDUA^{op} mediated enhanced expression and secretion of rIDUA in vitro. HeLa cell cultures were transfected in duplicates with 1 μg plasmid of ptrs-mCMV-hIDUA, ptrs-mCMV-hIDUA^{op} or ptrs-mCMV-ΔhIDUA^{op}. Controls were non-transfected HeLa cells. Cell lysates and media were assayed in duplicates for IDS activity at 48 hours post transfection. IDUA activity is expressed as units/mg protein (cells) or units/ml (media), 1 unit=1 nmol 4MU released/hr. NT:non-transfected HeLa cells; IDUA:HeLa cells transfected with ptrs-mCMV-hIDUA; IDUA-op: HeLa cells transfected with ptrs-mCMV-hIDUA^{op}; IDUA-op: HeLa cells transfected with ptrs-mCMV-ΔhIDUA^{op}.

FIG. 4 shows AAV-mediated correction of GAG storage in vitro in human MPS I skin fibroblasts (GM01257). 10 cm plates were transfected in duplicate with ptrs-mCMV-ΔhIDUA^{op} plasmid (7 μg/plate). 48 hours later, cell lysates were assayed in duplicate for GAG content. Media from the transfected cells were applied to non-treated GM01257 cells, and after 48 hours incubation, cell lysates were assayed in duplicate for GAG content. GAG contents are expressed as μg/5×10⁵ cells. Normal: healthy human fibroblasts (GM00969) cells; MPS I: non-treated GM01257 cells; AAV: GM01257 cells transfected with ptrs-mCMV-ΔhIDUA^{op} plasmid; AAV+media: GM01257 cells incubated with media from ptrs-mCMV-ΔhIDUA^{op} transfected GM01257 cells.

FIGS. 5A-5C show persistent rIDUA expression in the CNS and peripheral tissues after a systemic rAAV9-hIDUA gene delivery. MPS I mice were treated at ages 1 month or 6 months with an IV injection of A) 5×10¹² vg/kg, B) 1×10¹³ vg/kg, or C) 2×10¹² vg/kg, rAAV9-mCMV-hIDUA vector. Tissues were assayed for IDUA activity at 7 days, 1 month, 7 months, post vector injection (5A), or humane endpoint (5B), or 12 months post vector injection (5C). IDUA activity is expressed as units/mg protein, 1 unit=nmol 4MU released/hr. *No detectable IDUA activity in tissues of non-treated MPS I mice.

FIGS. 6A-6B show significant reduction of GAG content in the CNS and peripheral tissues in MPS I mice following a systemic rAAV9-hIDUA gene delivery. MPS I mice were treated at age 1 month with an IV injection of rAAV9-mCMV-hIDUA at A) 5×10¹² vg/kg or B) age 6 months at 1×10¹³ vg/kg. Tissues were assayed for GAG contents at 7 days, 1 month, 7 months post vector injection, or humane endpoint. GAG content is expressed as μg/mg wet tissue. *: p<0.05 vs. MPS I; #: p>0.05 vs. MPS I; +: p>0.05 vs. WT; \hat{ }: p<0.05 vs. WT.

FIGS. 7A-7D show correction of lysosomal storage and astrocytosis in MPS I mice following a systemic rAAV9-hIDUA vector delivery. MPS I mice were treated with an IV injection of 5×10¹² vg/kg rAAV9-mCMV-hIDUA at 1 month of age. Tissues were assayed at 7 months post-injection by immunofluorescence for LAMP 1 (red fluorescence) and GFP (green fluorescent protein). MPS I: non-treated MPS I controls; MPS I+AAV9: vector-treated MPS I mouse. A) brain: CTX: cerebral cortex; TH: thalamus: B) retina of eye: RET: retina; ON: outer nuclear layer; IN: inner nuclear layer; C) intestine: INT: small intestine; ME: muscularis externa; Small arrows: neurons of myenteric plexus; Large arrows: lamina propria; D) Other tissues: LIV: liver; LUNG: lung; HRT: heart; CIL: ciliary body of eye; IRIS: iris of eye; Scale bar: 50 μm.

FIGS. 8A-8C show significantly improved behavior performance and extended survival in MPS I mice after a systemic rAAV9-mCMV-hIDUA vector delivery. MPS I mice were treated with an IV injection of rAAV9-mCMV-hIDUA at age 1 month at 2×10¹² vg/kg or 5×10¹² vg/kg, or at age 6 months at 1×10¹³ vg/kg. Animals were tested for behavior performance in a hidden task in Morris water maze when they were 7 months old (n=8-29/group), and data were A) latency to find a hidden platform and B) swimming ability. C) Subsets of mice were observed for longevity.

FIGS. 9A-9H show differential biodistribution of systemically delivered rAAV9-mCMV-hIDUA vector in MPS I mice. MPS I mice were treated at age 1 month with an IV injection of 5×10¹² vg/kg rAAV9-mCMV-hIDUA, or at age 6 months with an IV injection of 1×10¹³ vg/kg rAAV9-mCMV-hIDUA. Tissues were assayed by qPCR for scAAV9-hIDUA vector genome at 1 month post vector injection, 7 months post-injection, or humane endpoint (n=1-9/group). *<0.001×10⁵ vg/μg gDNA were detected in tissues from non-treated MPS I mice.

FIGS. 10A-10B show quick restoration of IDUA activity and clearance of GAG contents in the CNS and peripheral organs in MPS I mice following an IV scAAV9-ΔhIDUA^{op} delivery. MPS I mice were treated at age 1 month with an IV injection of 2×10¹² vg/kg or 5×10¹² vg/kg scAAV9-mCMV-ΔhIDUA^{op} vector. At 1 month pi, tissues (n=4/group) were assayed for IDUA activity (A) and GAG contents (B). IDUA activity is expressed as units/mg protein, 1 unit=nmol 4MU released/hr. GAG content is expressed as μg/mg wet tissue. WT: wildtype mice; NT: non-treated MPS I mice. 2e12/5e12: vector-treated MPS I mice. \hat{ }: p<0.05 vs. WT; +: p>0.05 vs. WT; *: p<0.05 vs. NT; #: p>0.05 vs. NT.

FIG. 11 shows differential biodistribution of systemically delivered scAAV9-mCMV-ΔhIDUA^{op} vector in MPS I mice. MPS I mice were treated at age 1 month with an IV injection of 2×10¹² or 5×10¹² vg/kg scAAV9-mCMV-ΔhIDUA vector. Tissues (n=4/group) were assayed by qPCR for scAAV9-ΔhIDUA^{op} vector genome at 1 month pi. Data was vg/dg (diploid genome). *<0.002 vg/dg gDNA were detected in tissues from non-treated MPS I mice.

### DETAILED DESCRIPTION OF THE INVENTION

The present invention will now be described with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 CFR § 1.822 and established usage. See, e.g., PatentIn User Manual, 99-102 (November 1990) (U.S. Patent and Trademark Office).

Except as otherwise indicated, standard methods known to those skilled in the art may be used for the construction of recombinant parvovirus and AAV (rAAV) constructs, packaging vectors expressing the parvovirus Rep and/or Cap sequences, and transiently and stably transfected packaging cells. Such techniques are known to those skilled in the art. See. e.g., SAMBROOK et al., MOLECULAR CLONING: A LABORATORY MANUAL 4th Ed. (Cold Spring Harbor, NY, 2012); AUSUBEL et al., CURRENT PROTOCOLS IN MOLECULAR BIOLOGY (Green Publishing Associates, Inc. and John Wiley & Sons, Inc., New York).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination. To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed. For example, in particular embodiments the amino acid is not A, G or I; is not A; is not G or V; etc. as if each such possible disclaimer is expressly set forth herein.

### Definitions

The following terms are used in the description herein and the appended claims.

The singular forms “a” and “an” are intended to include the plural forms as well, unless the context clearly indicates otherwise.

Furthermore, the term “about,” as used herein when referring to a measurable value such as an amount of the length of a polynucleotide or polypeptide sequence, dose, time, temperature, and the like, is meant to encompass variations of 10%, 5%, 1%, 0.5%, or even 0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of” is to be interpreted as encompassing the recited materials or steps and those that do not materially affect the basic and novel characteristic(s) of the claimed invention (e.g., rAAV replication). Thus, the term “consisting essentially of” as used herein should not be interpreted as equivalent to “comprising.”

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence such that the function of the polynucleotide or polypeptide is not materially altered. The total of ten or less additional nucleotides or amino acids includes the total number of additional nucleotides or amino acids on both ends added together. The term “materially altered,” as applied to polynucleotides of the invention, refers to an increase or decrease in ability to express the encoded polypeptide of at least about 50% or more as compared to the expression level of a polynucleotide consisting of the recited sequence. The term “materially altered,” as applied to polypeptides of the invention, refers to an increase or decrease in enzymatic activity of at least about 50% or more as compared to the activity of a polypeptide consisting of the recited sequence.

The term “parvovirus” as used herein encompasses the family Parvoviridae, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, snake parvovirus, and B19 virus. Other autonomous parvoviruses are known to those skilled in the art. See, e.g., FIELDS et al., VIROLOGY, volume 2, chapter 69 (4th ed., Lippincott-Raven Publishers).

The genus Dependovirus contains the adeno-associated viruses (AAV), including but not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, avian AAV, bovine AAV, canine AAV, goat AAV, snake AAV, equine AAV, and ovine AAV. See, e.g., FIELDS et al., VIROLOGY, volume 2, chapter 69 (4th ed., Lippincott-Raven Publishers); and Table 1.

TABLE 1

| | GenBank Accession |
|---|---|
| Complete Genomes | Number |
| Adeno-associated virus 1 | NC_002077, AF063497 |
| Adeno-associated virus 2 | NC_001401 |
| Adeno-associated virus 3 | NC_001729 |
| Adeno-associated virus 3B | NC_001863 |
| Adeno-associated virus 4 | NC_001829 |
| Adeno-associated virus 5 | Y18065, AF085716 |
| Adeno-associated virus 6 | NC_001862 |
| Avian AAV ATCC VR-865 | AY186198, AY629583, |
| | NC_004828 |
| Avian AAV strain DA-1 | NC_006263, AY629583 |
| Bovine AAV | NC_005889, AY388617 |
| Clade A | |
| AAV1 | NC_002077, AF063497 |
| AAV6 | NC_001862 |
| Hu.48 | AY530611 |
| Hu 43 | AY530606 |
| Hu 44 | AY530607 |
| Hu 46 | AY530609 |
| Clade B | |
| Hu. 19 | AY530584 |
| Hu. 20 | AY530586 |
| Hu 23 | AY530589 |
| Hu22 | AY530588 |
| Hu24 | AY530590 |
| Hu21 | AY530587 |
| Hu27 | AY530592 |
| Hu28 | AY530593 |
| Hu 29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |
| Clade C | |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| Hu14 | AY530579 |
| (AAV9) | |
| Hu31 | AY530596 |
| Hu32 | AY530597 |
| Clonal Isolate | |
| AAV5 | Y18065, AF085716 |
| AAV 3 | NC_001729 |
| AAV 3B | NC_001863 |
| AAV4 | NC_001829 |
| Rh34 | AY243001 |
| Rh33 | AY243002 |
| Rh32 | AY243003 |

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, and any other AAV now known or later discovered. See, e.g., FIELDS et al., VIROLOGY, volume 2, chapter 69 (4th ed., Lippincott-Raven Publishers). A number of relatively new AAV serotypes and clades have been identified (See, e.g., Gao et al., (2004) J. Virol. 78:6381; Moris et al., (2004) Virol. 33-:375; and Table 1.

The parvovirus vectors, particles, and genomes of the present invention can be from, but are not limited to, AAV. The genomic sequences of various serotypes of AAV and the autonomous parvoviruses, as well as the sequences of the native ITRs, Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, AY631966, AX753250, EU285562, NC_001358, NC_001540, AF513851, AF513852 and AY530579; the disclosures of which are incorporated by reference herein for teaching parvovirus and AAV nucleic acid and amino acid sequences. See also, e.g., Bantel-Schaal et al., (1999) J. Virol. 73:939; Chiorini et al., (1997) J. Virol. 71:6823; Chiorini et al., (1999) J. Virol. 73:1309; Gao et al., (2002) Proc. Nat. Acad. Sci. USA 99:11854; Moris et al., (2004) Virol. 33-:375-383; Mori et al., (2004) Virol. 330:375; Muramatsu et al., (1996) Virol. 221:208; Ruffing et al., (1994) J. Gen. Virol. 75:3385; Rutledge et al., (1998) J. Virol. 72:309; Schmidt et al., (2008) J. Virol. 82:8911; Shade et al., (1986) J. Virol. 58:921; Srivastava et al., (1983) J. Virol. 45:555; Xiao et al., (1999) J. Virol. 73:3994; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1. An early description of the AAV1, AAV2 and AAV3 ITR sequences is provided by Xiao, X., (1996), “Characterization of Adeno-associated virus (AAV) DNA replication and integration,” Ph.D. Dissertation, University of Pittsburgh, Pittsburgh, PA (incorporated herein in its entirety).

The term “tropism” as used herein refers to entry of the virus into the cell, optionally and preferably followed by expression (e.g., transcription and, optionally, translation) of sequences carried by the viral genome in the cell, e.g., for a recombinant virus, expression of the heterologous nucleotide sequences(s). Those skilled in the art will appreciate that transcription of a heterologous nucleic acid sequence from the viral genome may not be initiated in the absence of trans-acting factors, e.g., for an inducible promoter or otherwise regulated nucleic acid sequence. In the case of AAV, gene expression from the viral genome may be from a stably integrated provirus, from a non-integrated episome, as well as any other form in which the virus may take within the cell.

As used herein, “transduction” of a cell by parvovirus or AAV refers to parvovirus/AAV-mediated transfer of genetic material into the cell. See, e.g., FIELDS et al., VIROLOGY, volume 2, chapter 69 (3d ed., Lippincott-Raven Publishers).

The terms “5′ portion” and “3′ portion” are relative terms to define a spatial relationship between two or more elements. Thus, for example, a “3′ portion” of a polynucleotide indicates a segment of the polynucleotide that is downstream of another segment. The term “3′ portion” is not intended to indicate that the segment is necessarily at the 3′ end of the polynucleotide, or even that it is necessarily in the 3′ half of the polynucleotide, although it may be. Likewise, a “5′ portion” of a polynucleotide indicates a segment of the polynucleotide that is upstream of another segment. The term “5′ portion” is not intended to indicate that the segment is necessarily at the 5′ end of the polynucleotide, or even that it is necessarily in the 5′ half of the polynucleotide, although it may be.

As used herein, the terms “protein” and “polypeptide” are used interchangeably and encompass both peptides and proteins, unless indicated otherwise.

As used herein, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA (including both naturally occurring and non-naturally occurring nucleotides), and can be either single or double stranded DNA sequences. The term polynucleotide, nucleotide sequence, or nucleic acid refers to a chain of nucleotides without regard to length of the chain. The nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

As used herein, “complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A: T) in the case of DNA, or adenine paired with uracil (A: U) in the case of RNA. For example, the sequence “A-G-T” binds to the complementary sequence “T-C-A.” It is understood that two polynucleotides may hybridize to each other even if they are not completely complementary to each other, provided that each has at least one region that is substantially complementary to the other.

As used herein, the term “substantially identical” or “corresponding to” means that two nucleic acid sequences have at least 60%, 70%, 80% or 90% sequence identity. In some embodiments, the two nucleic acid sequences can have at least 85%, 90%, 95%, 96%, 97%, 98%, 99% or 100% of sequence identity.

An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

The percent of sequence identity can be determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math., 2:482-489, 1981, Smith et al., Nucleic Acids Res. 11:2205-2220, 1983).

Useful methods for determining sequence identity are also disclosed in Guide to Huge Computers (Martin J. Bishop, ed., Academic Press, San Diego (1994)), and Carillo, H., and Lipton, D., (Applied Math 48:1073 (1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol. 215:403-410 (1990)); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

An “isolated polynucleotide” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence. An isolated polynucleotide that includes a gene is not a fragment of a chromosome that includes such gene, but rather includes the coding region and regulatory regions associated with the gene, but no additional genes naturally found on the chromosome.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., 90%, 92%, 95%, 98%, 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention.

As used herein, a “functional” polypeptide or “functional fragment” is one that substantially retains at least one biological activity normally associated with that peptide (e.g., degradation of glycosaminoglycans, heparan sulfates, and/or dermatan sulfates). In particular embodiments, the “functional” polypeptide or “functional fragment” substantially retains all of the activities possessed by the unmodified polypeptide. By “substantially retains” biological activity, it is meant that the peptide retains at least about 50%, 60%, 75%, 85%, 90%, 95%, 97%, 98%, 99%, or more, of the biological activity of the native polypeptide (and can even have a higher level of activity than the native peptide). A “non-functional” peptide is one that exhibits little or essentially no detectable biological activity normally associated with the peptide (e.g., at most, only an insignificant amount, e.g., less than about 10% or even 5%). Biological activities such as degradation of glycosaminoglycans, heparan sulfates, and/or dermatan sulfates can be measured using assays that are well known in the art and as described herein.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

As used herein, the term “modified,” as applied to a polynucleotide or polypeptide sequence, refers to a sequence that differs from a wild-type sequence due to one or more deletions, additions, substitutions, or any combination thereof.

As used herein, by “isolate” or “purify” (or grammatical equivalents) a virus vector, it is meant that the virus vector is at least partially separated from at least some of the other components in the starting material.

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject (e.g., in the case of MPS I, reduction in symptoms related to heart disease, skeletal problems, and central nervous system complications, prevention of respiratory tract infections, prevention of corneal clouding, or increase in survival time). Those skilled in the art will appreciate that the therapeutic effects need not be complete or curative, as long as some benefit is provided to the subject.

By the terms “treat,” “treating,” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing,” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

By the term “express” or “expression” of a polynucleotide coding sequence, it is meant that the sequence is transcribed, and optionally, translated. Typically, according to the present invention, expression of a coding sequence of the invention will result in production of the polypeptide of the invention. The entire expressed polypeptide or fragment can also function in intact cells without purification.

As used herein, the term “gene” refers to a nucleic acid molecule capable of being used to produce mRNA, antisense RNA, miRNA, and the like. Genes may or may not be capable of being used to produce a functional protein. Genes can include both coding and non-coding regions (e.g., introns, regulatory elements, promoters, enhancers, termination sequences and 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone or a plasmid.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged.

A “rAAV vector genome” or “rAAV genome” is an AAV genome (i.e., vDNA) that comprises one or more heterologous nucleic acid sequences. rAAV vectors generally require only the 145 base ITR in cis to generate virus. All other viral sequences are dispensable and may be supplied in trans (Muzyczka (1992) Curr. Topics Microbiol. Immunol. 158:97). Typically, the rAAV vector genome will only retain the one or more ITR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention the rAAV vector genome comprises at least one ITR sequence (e.g., AAV ITR sequence), optionally two ITRs (e.g., two AAV ITRs), which typically will be at the 5′ and 3′ ends of the vector genome and flank the heterologous nucleic acid, but need not be contiguous thereto. The ITRs can be the same or different from each other.

The term “terminal repeat” or “TR” includes any viral terminal repeat or synthetic sequence that forms a hairpin structure and functions as an inverted terminal repeat (i.e., mediates the desired functions such as replication, virus packaging, integration and/or provirus rescue, and the like). The ITR can be an AAV ITR or a non-AAV ITR. For example, a non-AAV ITR sequence such as those of other parvoviruses (e.g., canine parvovirus, bovine parvovirus, mouse parvovirus, porcine parvovirus, human parvovirus B-19) or the SV40 hairpin that serves as the origin of SV40 replication can be used as an ITR, which can further be modified by truncation, substitution, deletion, insertion and/or addition. Further, the ITR can be partially or completely synthetic, such as the “double-D sequence” as described in U.S. Pat. No. 5,478,745 to Samulski et al.

Parvovirus genomes have palindromic sequences at both their 5′ and 3′ ends. The palindromic nature of the sequences leads to the formation of a hairpin structure that is stabilized by the formation of hydrogen bonds between the complementary base pairs. This hairpin structure is believed to adopt a “Y” or a “T” shape. See, e.g., FIELDS et al., VIROLOGY, volume 2, chapters 69 & 70 (4th ed., Lippincott-Raven Publishers).

An “AAV inverted terminal repeat” or “AAV ITR” may be from any AAV, including but not limited to serotypes 1, 2, 3a, 3b, 4, 5, 6, 7, 8, 9, 10, 11, or 13, snake AAV, avian AAV, bovine AAV, canine AAV, equine AAV, ovine AAV, goat AAV, shrimp AAV, or any other AAV now known or later discovered (see, e.g., Table 1). An AAV ITR need not have the native terminal repeat sequence (e.g., a native AAV ITR sequence may be altered by insertion, deletion, truncation and/or missense mutations), as long as the terminal repeat mediates the desired functions, e.g., replication, virus packaging, persistence, and/or provirus rescue, and the like.

The virus vectors of the invention can further be “targeted” virus vectors (e.g., having a directed tropism) and/or a “hybrid” parvovirus (i.e., in which the viral ITRs and viral capsid are from different parvoviruses) as described in international patent publication WO 00/28004 and Chao et al., (2000) Mol. Therapy 2:619.

Further, the viral capsid or genomic elements can contain other modifications, including insertions, deletions and/or substitutions.

The term “template” or “substrate” is used herein to refer to a polynucleotide sequence that may be replicated to produce the parvovirus viral DNA. For the purpose of vector production, the template will typically be embedded within a larger nucleotide sequence or construct, including but not limited to a plasmid, naked DNA vector, bacterial artificial chromosome (BAC), yeast artificial chromosome (YAC) or a viral vector (e.g., adenovirus, herpesvirus, Epstein-Barr Virus, AAV, baculoviral, retroviral vectors, and the like). Alternatively, the template may be stably incorporated into the chromosome of a packaging cell.

As used herein, parvovirus or AAV “Rep coding sequences” indicate the nucleic acid sequences that encode the parvoviral or AAV non-structural proteins that mediate viral replication and the production of new virus particles. The parvovirus and AAV replication genes and proteins have been described in, e.g., FIELDS et al., VIROLOGY, volume 2, chapters 69 & 70 (4th ed., Lippincott-Raven Publishers).

The “Rep coding sequences” need not encode all of the parvoviral or AAV Rep proteins. For example, with respect to AAV, the Rep coding sequences do not need to encode all four AAV Rep proteins (Rep78, Rep 68, Rep52 and Rep40), in fact, it is believed that AAV5 only expresses the spliced Rep68 and Rep40 proteins. In representative embodiments, the Rep coding sequences encode at least those replication proteins that are necessary for viral genome replication and packaging into new virions. The Rep coding sequences will generally encode at least one large Rep protein (i.e., Rep78/68) and one small Rep protein (i.e., Rep52/40). In particular embodiments, the Rep coding sequences encode the AAV Rep78 protein and the AAV Rep52 and/or Rep40 proteins. In other embodiments, the Rep coding sequences encode the Rep68 and the Rep52 and/or Rep40 proteins. In a still further embodiment, the Rep coding sequences encode the Rep68 and Rep52 proteins, Rep68 and Rep40 proteins, Rep78 and Rep52 proteins, or Rep78 and Rep40 proteins.

As used herein, the term “large Rep protein” refers to Rep68 and/or Rep78. Large Rep proteins of the claimed invention may be either wild-type or synthetic. A wild-type large Rep protein may be from any parvovirus or AAV, including but not limited to serotypes 1, 2, 3a, 3b, 4, 5, 6, 7, 8, 9, 10, 11, or 13, or any other AAV now known or later discovered (see, e.g., Table 1). A synthetic large Rep protein may be altered by insertion, deletion, truncation and/or missense mutations.

Those skilled in the art will further appreciate that it is not necessary that the replication proteins be encoded by the same polynucleotide. For example, for MVM, the NS-1 and NS-2 proteins (which are splice variants) may be expressed independently of one another. Likewise, for AAV, the p19 promoter may be inactivated and the large Rep protein(s) expressed from one polynucleotide and the small Rep protein(s) expressed from a different polynucleotide. Typically, however, it will be more convenient to express the replication proteins from a single construct. In some systems, the viral promoters (e.g., AAV p19 promoter) may not be recognized by the cell, and it is therefore necessary to express the large and small Rep proteins from separate expression cassettes. In other instances, it may be desirable to express the large Rep and small Rep proteins separately, i.e., under the control of separate transcriptional and/or translational control elements. For example, it may be desirable to control expression of the large Rep proteins, so as to decrease the ratio of large to small Rep proteins. In the case of insect cells, it may be advantageous to down-regulate expression of the large Rep proteins (e.g., Rep78/68) to avoid toxicity to the cells (see, e.g., Urabe et al., (2002) Human Gene Therapy 13:1935).

As used herein, the parvovirus or AAV “cap coding sequences” encode the structural proteins that form a functional parvovirus or AAV capsid (i.e., can package DNA and infect target cells). Typically, the cap coding sequences will encode all of the parvovirus or AAV capsid subunits, but less than all of the capsid subunits may be encoded as long as a functional capsid is produced. Typically, but not necessarily, the cap coding sequences will be present on a single nucleic acid molecule.

The capsid structure of autonomous parvoviruses and AAV are described in more detail in BERNARD N. FIELDS et al., VIROLOGY, volume 2, chapters 69 & 70 (4th ed., Lippincott-Raven Publishers).

### Parvovirus Vectors Expressing IDUA

To address the unmet needs for optimal therapeutic potential, the inventors have developed novel self-complementary (sc) AAV9 gene therapy products expressing human IDUA gene (hIDUA) for the treatment of MPS I. To accommodate the limited packaging capacity of scAAV vectors, the disclosed product was constructed using a 228 bp truncated miniature CMV promoter (mCMV), a 16 bp soluble neuropilin-1 (sNRP-1) Poly A signal, and miniaturized hIDUA^{op} cDNA (ΔhIDUA^{op}), which enabled the successful packaging of scAAV-ΔhIDUA^{op} Miniaturized hIDUA^{op} (ΔhIDUA^{op}) was constructed by deleting 15 bp of non-coding sequence from near the 3′-end. These changes in the ΔhIDUA^{op} construct have resulted in an 8-fold increase in the expression and 10-fold increase in the secretion of rIDUA in comparison to the inventors' previous ssAAV-hIDUA^{op} construct, which increased by approximately 25% in total IDUA activity compared to a vector comprising a WT IDUA encoding sequence (ptrs-mCMV-hIDUA). This side-by-side comparison was made using plasmid transfection to eliminate any effects of differential transduction efficiency and suggests that the 15 bp deletion itself had a beneficial effect on expression levels. This scAAV-ΔhIDUA^{op} is the first scAAV-IDUA vector to date, and because the scAAV vector bypasses the requirement for DNA synthesis by the host cells to convert the single-stranded (ss) vg into active double-stranded DNA, there may be further transduction benefits which will independently multiply the effects of the changes in the transgene construct itself. These results support the great therapeutic potential of scAAV9-hIDUA^{op} via systemic delivery for treating MPS I in humans.

The present invention provides parvovirus vectors, e.g., AAV vectors, that comprise a nucleotide sequence encoding IDUA and are capable of expressing IDUA in a subject.

One aspect of the invention relates to a recombinant nucleic acid comprising a nucleotide sequence encoding human IDUA or a functional fragment thereof, wherein the nucleotide sequence has been codon-optimized for expression in human cells. In some embodiments, the nucleic acid comprises a nucleotide sequence that is at least 90% identical to SEQ ID NO:1, e.g., at least 91%, 92%, 93%, 94%, 95%, 96%, 97%, 98%, 99%, 99.5%, or 99.9% identical to SEQ ID NO:1. In some embodiments, the nucleic acid comprises, consists essentially of, or consists of the nucleotide sequence of SEQ ID NO:1.

```
SEQ ID NO: 1
      1 ATGCGACCTC TGAGGCCACG GGCTGCGTTG TTGGCTCTTT TGGCGTCCCT
    51 GTTGGCCGCA CCGCCTGTCG CTCCAGCAGA AGCACCCCAC CTCGTGCATG
  101 TTGATGCAGC ACGAGCACTG TGGCCGCTTC GGAGGTTTTG GAGGTCAACG
  151 GGGTTCTGTC CTCCATTGCC ACACTCTCAA GCGGATCAGT ATGTGTTGTC
  201 TTGGGATCAG CAACTGAACC TGGCCTATGT CGGTGCCGTA CCTCATCGCG
  251 GAATTAAACA GGTTCGAACC CATTGGCTCC TTGAGTTGGT TACGACACGG
  301 GGCAGCACAG GAAGGGGACT CTCTTACAAT TTCACTCACT TGGACGGATA
  351 CCTTGACCTC CTGCGAGAGA ATCAGCTGCT TCCCGGTTTC GAGTTGATGG
  401 GGTCAGCTAG CGGACACTTC ACTGATTTCG AGGATAAGCA ACAAGTTTTT
  451 GAGTGGAAAG ACCTCGTGTC ATCACTTGCC CGGAGGTATA TAGGGCGCTA
  501 CGGGCTTGCG CACGTAAGTA AGTGGAACTT TGAGACGTGG AATGAGCCTG
  551 ATCACCACGA TTTTGACAAT GTTTCTATGA CGATGCAGGG CTTTCTCAAC
  601 TACTATGACG CGTGTTCTGA AGGACTGAGA GCAGCCAGTC CGGCACTCCG
  651 CCTGGGGGGC CCAGGGGACT CCTTCCACAC ACCGCCTCGG TCCCCGCTTT
  701 CTTGGGGGCT GTTGCGACAC TGTCATGACG GTACGAACTT TTTTACAGGA
  751 GAAGCGGGCG TCCGCCTCGA TTATATTTCT CTGCATCGAA AAGGTGCGAG
  801 GTCCTCAATC TCTATCCTGG AGCAGGAGAA GGTCGTGGCA CAACAAATTA
  851 GACAACTCTT TCCTAAATTT GCCGACACCC CTATCTATAA TGATGAGGCC
  901 GACCCTCTTG TGGGTTGGTC ACTTCCGCAA CCCTGGCGGG CGGATGTGAC
  951 ATATGCAGCA ATGGTAGTCA AAGTGATCGC GCAACATCAA AACTTGCTCC
1001 TGGCAAACAC GACATCCGCT TTTCCGTATG CCTTGCTTTC AAACGATAAC
1051 GCTTTCCTTT CATACCACCC GCATCCCTTT GCACAGCGAA CCCTCACAGC
1101 GCGGTTCCAG GTCAATAACA CACGACCTCC TCACGTCCAG TTGTTGAGAA
1151 AACCTGTGCT TACTGCCATG GGACTTCTGG CACTTCTCGA CGAGGAACAG
1201 CTGTGGGCGG AGGTATCTCA AGCAGGGACT GTATTGGATA GCAACCACAC
1251 CGTAGGGGTC CTCGCCTCCG CGCACAGGCC ACAAGGACCT GCTGATGCTT
1301 GGCGAGCTGC AGTTCTGATC TACGCCAGTG ACGACACTAG GGCCCATCCG
1351 AACAGATCAG TCGCGGTGAC GCTGAGGCTC CGCGGAGTGC CCCCTGGTCC
1401 CGGTCTCGTT TACGTCACTC GCTACCTCGA CAACGGACTC TGTTCTCCGG
1451 ATGGCGAGTG GAGAAGACTT GGACGGCCTG TTTTCCCGAC CGCGGAACAA
1501 TTCCGGCGAA TGAGGGCCGC TGAGGACCCA GTAGCGGCAG CGCCAAGACC
1551 TCTCCCCGCT GGGGGCCGCC TCACCCTTAG ACCCGCTCTC AGGCTCCCCA
1601 GTCTTCTCTT GGTACACGTA TGTGCCCGAC CTGAGAAACC CCCTGGCCAG
1651 GTCACTCGCT TGCGAGCTCT TCCTTTGACT CAAGGACAAC TTGTCCTCGT
1701 TTGGAGCGAC GAGCATGTGG GCTCTAAATG CCTCTGGACT TATGAAATAC
1751 AGTTCTCACA AGATGGCAAA GCATACACCC CGGTATCTAG GAAACCCTCA
1801 ACGTTCAACC TGTTTGTATT CTCCCCCGAC ACGGGAGCCG TGTCAGGCTC
1851 CTACCGAGTT CGGGCCTTGG ACTACTGGGC TAGGCCCGGC CCCTTCTCCG
1901 ATCCAGTCCC ATATTTGGAG GTCCCTGTTC CGCGCGGACC CCCCAGCCCG
1951 GGCAATCCCT GA
```

Another aspect of the invention relates to a recombinant nucleic acid comprising a nucleotide sequence encoding human IDUA, wherein the nucleotide sequence has been truncated (15 non-coding nucleotides are removed from near the 3′ end (e.g., the 15 nucleotides ending 15 nucleotides upstream of the stop codon)) for expression in human cells. As used herein, the phrase “near the 3′ end” means within 50 nucleotides of the stop codon, e.g., within 40 or 30 nucleotides of the stop codon. In some embodiments, the nucleic acid comprises a nucleotide sequence that is at least 90% identical to SEQ ID NO:2, e.g., at least 91%, 92%, 93%, 94%, 95%, 96%, 97%, 98%, 99%, 99.5%, or 99.9% identical to SEQ ID NO:2. In some embodiments, the nucleic acid comprises, consists essentially of, or consists of the nucleotide sequence of SEQ ID NO:2.

```
SEQ ID NO: 2
      1 ATGCGTCCCC TGCGCCCCCG CGCCGCGCTG CTGGCGCTCC TGGCCTCGCT
    51 CCTGGCCGCG CCCCCGGTGG CCCCGGCCGA GGCCCCGCAC CTGGTGCATG
  101 TGGACGCGGC CCGCGCGCTG TGGCCCCTGC GGCGCTTCTG GAGGAGCACA
  151 GGCTTCTGCC CCCCGCTGCC ACACAGCCAG GCTGACCAGT ACGTCCTCAG
  201 CTGGGACCAG CAGCTCAACC TCGCCTATGT GGGCGCCGTC CCTCACCGCG
  251 GCATCAAGCA GGTCCGGACC CACTGGCTGC TGGAGCTTGT CACCACCAGG
  301 GGGTCCACTG GACGGGGCCT GAGCTACAAC TTCACCCACC TGGACGGGTA
  351 CCTGGACCTT CTCAGGGAGA ACCAGCTCCT CCCAGGGTTT GAGCTGATGG
  401 GCAGCGCCTC GGGCCACTTC ACTGACTTTG AGGACAAGCA GCAGGTGTTT
  451 GAGTGGAAGG ACTTGGTCTC CAGCCTGGCC AGGAGATACA TCGGTAGGTA
  501 CGGACTGGCG CATGTTTCCA AGTGGAACTT CGAGACGTGG AATGAGCCAG
  551 ACCACCACGA CTTTGACAAC GTCTCCATGA CCATGCAAGG CTTCCTGAAC
  601 TACTACGATG CCTGCTCGGA GGGTCTGCGC GCCGCCAGCC CCGCCCTGCG
  651 GCTGGGAGGC CCCGGCGACT CCTTCCACAC CCCACCGCGA TCCCCGCTGA
  701 GCTGGGGCCT CCTGCGCCAC TGCCACGACG GTACCAACTT CTTCACTGGG
  751 GAGGGGGGCG TGCGGCTGGA CTACATCTCC CTCCACAGGA AGGGTGCGCG
  801 CAGCTCCATC TCCATCCTGG AGCAGGAGAA GGTCGTCGCG CAGCAGATCC
  851 GGCAGCTCTT CCCCAAGTTC GCGGACACCC CCATTTACAA CGACGAGGCG
  901 GACCCGCTGG TGGGCTGGTC CCTGCCACAG CCGTGGAGGG CGGACGTGAC
  951 CTACGCGGCC ATGGTGGTGA AGGTCATCGC GCAGCATCAG AACCTGCTAC
1001 TGGCCAACAC CACCTCCGCC TTCCCCTACG CGCTCCTGAG CAACGACAAT
1051 GCCTTCCTGA GCTACCACCC GCACCCCTTC GCGCAGCGCA CGCTCACCGC
1101 GCGCTTCCAG GTCAACAACA CCCGCCCGCC GCACGTGCAG CTGTTGCGCA
1151 AGCCGGTGCT CACGGCCATG GGGCTGCTGG CGCTGCTGGA TGAGGAGCAG
1201 CTCTGGGCCG AAGTGTCGCA GGCCGGGACC GTCCTGGACA GCAACCACAC
1251 GGTGGGCGTC CTGGCCAGCG CCCACCGCCC CCAGGGCCCG GCCGACGCCT
1301 GGCGCGCCGC GGTGCTGATC TACGCGAGCG ACGACACCCG CGCCCACCCC
1351 AACCGCAGCG TCGCGGTGAC CCTGCGGCTG CGCGGGGTGC CCCCCGGCCC
1401 GGGCCTGGTC TACGTCACGC GCTACCTGGA CAACGGGCTC TGCAGCCCCG
1451 ACGGCGAGTG GCGGCGCCTG GGCCGGCCCG TCTTCCCCAC GGCAGAGCAG
1501 TTCCGGCGCA TGCGCGCGGC TGAGGACCCG GTGGCCGCGG CGCCCCGCCC
1551 CTTACCCGCC GGCGGCCGCC TGACCCTGCG CCCCGCGCTG CGGCTGCCGT
1601 CGCTTTTGCT GGTGCACGTG TGTGCGCGCC CCGAGAAGCC GCCCGGGCAG
1651 GTCACGCGGC TCCGCGCCCT GCCCCTGACC CAAGGGCAGC TGGTTCTGGT
1701 CTGGTCGGAT GAACACGTGG GCTCCAAGTG CCTGTGGACA TACGAGATCC
1751 AGTTCTCTCA GGACGGTAAG GCGTACACCC CGGTCAGCAG GAAGCCATCG
1801 ACCTTCAACC TCTTTGTGTT CAGCCCAGAC ACAGGTGCTG TCTCTGGCTC
1851 CTACCGAGTT CGAGCCCTGG ACTACTGGGC CCGACCAGGC CCCTTCTCGG
1901 ACCCTGTGCC GTACCTGGAG GTCCCTGTGT CCCCGGGCAA TCCATGA
```

Another aspect of the invention relates to a recombinant nucleic acid comprising a nucleotide sequence encoding human IDUA, wherein the nucleotide sequence has been codon-optimized and truncated (15 non-coding nucleotides are removed from near the 3′ end) for expression in human cells. In some embodiments, the nucleic acid comprises a nucleotide sequence that is at least 90% identical to SEQ ID NO:3, e.g., at least 91%, 92%, 93%, 94%, 95%, 96%, 97%, 98%, 99%, 99.5%, or 99.9% identical to SEQ ID NO:3. In some embodiments, the nucleic acid comprises, consists essentially of, or consists of the nucleotide sequence of SEQ ID NO:3.

```
SEQ ID NO: 3
      1 ATGCGACCTC TGAGGCCACG GGCTGCGTTG TTGGCTCTTT TGGCGTCCCT
    51 GTTGGCCGCA CCGCCTGTCG CTCCAGCAGA AGCACCCCAC CTCGTGCATG
  101 TTGATGCAGC ACGAGCACTG TGGCCGCTTC GGAGGTTTTG GAGGTCAACG
  151 GGGTTCTGTC CTCCATTGCC ACACTCTCAA GCGGATCAGT ATGTGTTGTC
  201 TTGGGATCAG CAACTGAACC TGGCCTATGT CGGTGCCGTA CCTCATCGCG
  251 GAATTAAACA GGTTCGAACC CATTGGCTCC TTGAGTTGGT TACGACACGG
  301 GGCAGCACAG GAAGGGGACT CTCTTACAAT TTCACTCACT TGGACGGATA
  351 CCTTGACCTC CTGCGAGAGA ATCAGCTGCT TCCCGGTTTC GAGTTGATGG
  401 GGTCAGCTAG CGGACACTTC ACTGATTTCG AGGATAAGCA ACAAGTTTTT
  451 GAGTGGAAAG ACCTCGTGTC ATCACTTGCC CGGAGGTATA TAGGGCGCTA
  501 CGGGCTTGCG CACGTAAGTA AGTGGAACTT TGAGACGTGG AATGAGCCTG
  551 ATCACCACGA TTTTGACAAT GTTTCTATGA CGATGCAGGG CTTTCTCAAC
  601 TACTATGACG CGTGTTCTGA AGGACTGAGA GCAGCCAGTC CGGCACTCCG
  651 CCTGGGGGGC CCAGGGGACT CCTTCCACAC ACCGCCTCGG TCCCCGCTTT
  701 CTTGGGGGCT GTTGCGACAC TGTCATGACG GTACGAACTT TTTTACAGGA
  751 GAAGCGGGCG TCCGCCTCGA TTATATTTCT CTGCATCGAA AAGGTGCGAG
  801 GTCCTCAATC TCTATCCTGG AGCAGGAGAA GGTCGTGGCA CAACAAATTA
  851 GACAACTCTT TCCTAAATTT GCCGACACCC CTATCTATAA TGATGAGGCC
  901 GACCCTCTTG TGGGTTGGTC ACTTCCGCAA CCCTGGCGGG CGGATGTGAC
  951 ATATGCAGCA ATGGTAGTCA AAGTGATCGC GCAACATCAA AACTTGCTCC
1001 TGGCAAACAC GACATCCGCT TTTCCGTATG CCTTGCTTTC AAACGATAAC
1051 GCTTTCCTTT CATACCACCC GCATCCCTTT GCACAGCGAA CCCTCACAGC
1101 GCGGTTCCAG GTCAATAACA CACGACCTCC TCACGTCCAG TTGTTGAGAA
1151 AACCTGTGCT TACTGCCATG GGACTTCTGG CACTTCTCGA CGAGGAACAG
1201 CTGTGGGCGG AGGTATCTCA AGCAGGGACT GTATTGGATA GCAACCACAC
1251 CGTAGGGGTC CTCGCCTCCG CGCACAGGCC ACAAGGACCT GCTGATGCTT
1301 GGCGAGCTGC AGTTCTGATC TACGCCAGTG ACGACACTAG GGCCCATCCG
1351 AACAGATCAG TCGCGGTGAC GCTGAGGCTC CGCGGAGTGC CCCCTGGTCC
1401 CGGTCTCGTT TACGTCACTC GCTACCTCGA CAACGGACTC TGTTCTCCGG
1451 ATGGCGAGTG GAGAAGACTT GGACGGCCTG TTTTCCCGAC CGCGGAACAA
1501 TTCCGGCGAA TGAGGGCCGC TGAGGACCCA GTAGCGGCAG CGCCAAGACC
1551 TCTCCCCGCT GGGGGCCGCC TCACCCTTAG ACCCGCTCTC AGGCTCCCCA
1601 GTCTTCTCTT GGTACACGTA TGTGCCCGAC CTGAGAAACC CCCTGGCCAG
1651 GTCACTCGCT TGCGAGCTCT TCCTTTGACT CAAGGACAAC TTGTCCTCGT
1701 TTGGAGCGAC GAGCATGTGG GCTCTAAATG CCTCTGGACT TATGAAATAC
1751 AGTTCTCACA AGATGGCAAA GCATACACCC CGGTATCTAG GAAACCCTCA
1801 ACGTTCAACC TGTTTGTATT CTCCCCCGAC ACGGGAGCCG TGTCAGGCTC
1851 CTACCGAGTT CGGGCCTTGG ACTACTGGGC TAGGCCCGGC CCCTTCTCCG
1901 ATCCAGTCCC ATATTTGGAG GTCCCTGTTA GCCCGGGCAA TCCCTGA
```

Methods of codon optimizing a nucleotide sequence to maximize expression in an organism are well known in the art and can be carried out using software available to the public. The wild-type sequence of human IDUA is known in the art and can be found in databases such as GenBank. Examples of human IDUA accession numbers include A26494, AK291816, and AH002600, incorporated by reference herein in their entirety.

The invention also provides a viral vector genome comprising the IDUA nucleic acid of the invention. In certain embodiments, the IDUA nucleic acid is the wild-type human IDUA sequence or the codon-optimized sequence. The viral vector genome may be a parvovirus vector genome, e.g., an AAV vector genome. In one embodiment, a scAAV vector has a mCMV promoter, a sNRP-1 Poly A signal, and hIDUA^{op}, which is codon optimized and miniaturized by deleting 15 nucleotides of non-conserved sequence (ΔhIDUA^{op}) near the 3′-end and has a sequence at least 90% identical to SEQ ID NO:4 (ptrs-mCMV-ΔhIDUA^{op}), e.g., at least 91%, 92%, 93%, 94%, 95%, 96%, 97%, 98%, 99%, 99.5%, or 99.9% identical to SEQ ID NO:4.

```
SEQ ID NO: 4
      1 CAGCAGCTGC GCGCTCGCTC GCTCACTGAG GCCGCCCGGG CAAAGCCCGG
    51 GCGTCGGGCG ACCTTTGGTC GCCCGGCCTC AGTGAGCGAG CGAGCGCGCA
  101 GAGAGGGAGT GGGGTTATTT CGTATTTTAT TTAGATCTGA TGGTCAGGGA
  151 TTGCCCGGGC TAACAGGGAC CTCCAAATAT GGGACTGGAT CGGAGAAGGG
  201 GCCGGGCCTA GCCCAGTAGT CCAAGGCCCG AACTCGGTAG GAGCCTGACA
  251 CGGCTCCCGT GTCGGGGGAG AATACAAACA GGTTGAACGT TGAGGGTTTC
  301 CTAGATACCG GGGTGTATGC TTTGCCATCT TGTGAGAACT GTATTTCATA
  351 AGTCCAGAGG CATTTAGAGC CCACATGCTC GTCGCTCCAA ACGAGGACAA
  401 GTTGTCCTTG AGTCAAAGGA AGAGCTCGCA AGCGAGTGAC CTGGCCAGGG
  451 GGTTTCTCAG GTCGGGCACA TACGTGTACC AAGAGAAGAC TGGGGAGCCT
  501 GAGAGCGGGT CTAAGGGTGA GGCGGCCCCC AGCGGGGAGA GGTCTTGGCG
  551 CTGCCGCTAC TGGGTCCTCA GCGGCCCTCA TTCGCCGGAA TTGTTCCGCG
  601 GTCGGGAAAA CAGGCCGTCC AAGTCTTCTC CACTCGCCAT CCGGAGAACA
  651 GAGTCCGTTG TCGAGGTAGC GAGTGACGTA AACGAGACCG GGACCAGGGG
  701 GCACTCCGCG GAGCCTCAGC GTCACCGCGA CTGATCTGTT CGGATGGGCC
  751 CTAGTGTCGT CACTGGCGTA GATCAGAACT GCAGCTCGCC AAGCATCAGC
  801 AGGTCCTTGT GGCCTGTGCG CGGAGGCGAG GACCCCTACG GTGTGGTTGC
  851 TATCCAATAC AGTCCCTGCT TGAGATACCT CCGCCCACAG CTGTTCCTCG
  901 TCGAGAAGTG CCAGAAGTCC CATGGCAGTA AGCACAGGTT TTCTCAACAA
  951 CTGGACGTGA GGAGGTCGTG TGTTATTGAC CTGGAACCGC GCTGTGAGGG
1001 TTCGCTGTGC AAAGGGATGC GGGTGGTATG AAAGGAAAGC GTTATCGTTT
1051 GAAAGCAAGG CATACGGAAA AGCGGATGTC GTGTTTGCCA GGAGCAAGTT
1101 TTGATGTTGC GCGATCACTT TGACTACCAT TGCTGCATAT GTCACATCCG
1151 CCCGCCAGGG TTGCGGAAGT GACCAACCCA CAAGAGGGTC GGCCTCATCA
1201 TTATAGATAG GGGTGTCGGC AAATTTAGGA AAGAGTTGTC TAATTTGTTG
1251 TGCCACGACC TTCTCCTGCT CCAGGATAGA GATTGAGGAC CTCGCACCTT
1301 TTCGATGCAG AGAAATATAA TCGAGGCGGA CGCCCGCTTC TCCTGTAAAA
1351 AAGTTCGTAC CGTCATGACA GTGTCGCAAC AGCCCCCAAG AAAGCGGGGA
1401 CCGAGGCGGT GTGTGGAAGG AGTCCCCTGG GCCCCCCAGG CGGAGTGCCG
1451 GACTGGCTGC TCTCAGTCCT TCAGAACACG CGTCATAGTA GTTGAGAAAG
1501 CCCTGCATCG TCATAGAAAC ATTGTCAAAA TCGTGGTGAT CAGGCTCATT
1551 CCACGTCTCA AAGTTCCACT TACTTACGTG CGCAAGCCCG TAGCGCCCTA
1601 TATACCTCCG GGCAAGTGAT GACACGAGGT CTTTCCACTC AAAAACTTGT
1651 TGCTTATCCT CGAAATCAGT GAAGTGTCCG CTAGCTGACC CCATCAACTC
1701 GAAACCGGGA AGCAGCTGAT TCTCTCGCAG GAGGTCAAGG TATCCGTCCA
1751 AGTGAGTGAA ATTGTAAGAG AGTCCCCTTC CTGTGCTGCC CCGTGTCGTA
1801 ACCAACTCAA GGAGCCAATG GGTTCGAACC TGTTTAATTC CGCGATGAGG
1851 TACGGCACCG ACATAGGCCA GGTTCAGTTG CTGATCCCAA GACAACACAT
1901 ACTGATCCGC TTGAGAGTGT GGCAATGGAG GACAGAACCC CGTTGACCTC
1951 CAAAACCTCC GAAGCGGCCA CAGTGCTCGT GCTGCATCAA CATGCACGAG
2001 GTGGGGTGCT TCTGCTGGAG CGACAGGCGG TGCGGCCAAC AGGGACGCCA
2051 AAAGAGCCAA CAACGCAGCC CGTGGCCTCA GAGGTCGCAT GGCGACCGGT
2101 CGAGCGGTTC ACTAAACGAG CTCTGCTTAT ATAGACCTCC CACCGTACAC
2151 GCCTACCGCC CATTTGCGTC AATGGGGCGG AGTTGTTACG ACATTTTGGA
2201 AAGTCCCGTT GATTTTGGTG CCAAAACAAA CTCCCATTGA CGTCAATGGG
2251 GTGGAGACTT GGAAATCCCC GTGAGTCCGG GGGTCGTTGG GCGGTCAGCC
2301 AGGCGGGCCA TTTACCGTAA GTTATGTAAC GAAAGCTTCC TAGGAACCCC
2351 TAGTGATGGA GTTGGCCACT CCCTCTCTGC GCGCTCGCTC GCTCACTGAG
2401 GCCGGGCGAC CAAAGGTCGC CCGACGCCCG GGCTTTGCCC GGGCGGCCTC
2451 AGTGAGCGAG CGAGCGCGCA GCTGGCGTAA TAGCGAAGAG GCCCGCACCG
2501 ATCGCCCTTC CCAACAGTTG CGCAGCCTGA ATGGCGAATG GCGATTCCGT
2551 TGCAATGGCT GGCGGTAATA TTGTTCTGGA TATTACCAGC AAGGCCGATA
2601 GTTTGAGTTC TTCTACTCAG GCAAGTGATG TTATTACTAA TCAAAGAAGT
2651 ATTGCGACAA CGGTTAATTT GCGTGATGGA CAGACTCTTT TACTCGGTGG
2701 CCTCACTGAT TATAAAAACA CTTCTCAGGA TTCTGGCGTA CCGTTCCTGT
2751 CTAAAATCCC TTTAATCGGC CTCCTGTTTA GCTCCCGCTC TGATTCTAAC
2801 GAGGAAAGCA CGTTATACGT GCTCGTCAAA GCAACCATAG TACGCGCCCT
2851 GTAGCGGCGC ATTAAGCGCG GCGGGTGTGG TGGTTACGCG CAGCGTGACC
2901 GCTACACTTG CCAGCGCCCT AGCGCCCGCT CCTTTCGOTT TCTTCCCTTC
2951 CTTTCTCGCC ACGTTCGCCG GCTTTCCCCG TCAAGCTCTA AATCGGGGGC
3001 TCCCTTTAGG GTTCCGATTT AGTGCTTTAC GGCACCTCGA CCCCAAAAAA
3051 CTTGATTAGG GTGATGGTTC ACGTAGTGGG CCATCGCCCT GATAGACGGT
3101 TTTTCGCCCT TTGACGTTGG AGTCCACGTT CTTTAATAGT GGACTCTTGT
3151 TCCAAACTGG AACAACACTC AACCCTATCT CGGTCTATTC TTTTGATTTA
3201 TAAGGGATTT TGCCGATTTC GGCCTATTGG TTAAAAAATG AGCTGATTTA
3251 ACAAAAATTT AACGCGAATT TTAACAAAAT ATTAACGCTT ACAATTTAAA
3301 TATTTGCTTA TACAATCTTC CTGTTTTTGG GGCTTTTCTG ATTATCAACC
3351 GGGGTACATA TGATTGACAT GCTAGTTTTA CGATTACCGT TCATCGATTC
3401 TCTTGTTTGC TCCAGACTCT CAGGCAATGA CCTGATAGCC TTTGTAGAGA
3451 CCTCTCAAAA ATAGCTACCC TCTCCGGCAT GAATTTATCA GCTAGAACGG
3501 TTGAATATCA TATTGATGGT GATTTGACTG TCTCCGGCCT TTCTCACCCG
3551 TTTGAATCTT TACCTACACA TTACTCAGGC ATTGCATTTA AAATATATGA
3601 GGGTTCTAAA AATTTTTATC CTTGCGTTGA AATAAAGGCT TCTCCCGCAA
3651 AAGTATTACA GGGTCATAAT GTTTTTGGTA CAACCGATTT AGCTTTATGC
3701 TCTGAGGCTT TATTGCTTAA TTTTGCTAAT TCTTTGCCTT GCCTGTATGA
3751 TTTATTGGAT GTTGGAATCG CCTGATGCGG TATTTTCTCC TTACGCATCT
3801 GTGCGGTATT TCACACCGCA TATGGTGCAC TCTCAGTACA ATCTGCTCTG
3851 ATGCCGCATA GTTAAGCCAG CCCCGACACC CGCCAACACC CGCTGACGCG
3901 CCCTGACGGG CTTGTCTGCT CCCGGCATCC GCTTACAGAC AAGCTGTGAC
3951 CGTCTCCGGG AGCTGCATGT GTCAGAGGTT TTCACCGTCA TCACCGAAAC
4001 GCGCGAGACG AAAGGGCCTC GTGATACGCC TATTTTTATA GGTTAATGTC
4051 ATGATAATAA TGGTTTCTTA GACGTCAGGT GGCACTTTTC GGGGAAATGT
4101 GCGCGGAACC CCTATTTGTT TATTTTTCTA AATACATTCA AATATGTATC
4151 CGCTCATGAG ACAATAACCC TGATAAATGC TTCAATAATA TTGAAAAAGG
4201 AAGAGTATGA GTATTCAACA TTTCCGTGTC GCCCTTATTC CCTTTTTTGC
4251 GGCATTTTGC CTTCCTGTTT TTGCTCACCC AGAAACGCTG GTGAAAGTAA
4301 AAGATGCTGA AGATCAGTTG GGTGCACGAG TGGGTTACAT CGAACTGGAT
4351 CTCAACAGCG GTAAGATCCT TGAGAGTTTT CGCCCCGAAG AACGTTTTCC
4401 AATGATGAGC ACTTTTAAAG TTCTGCTATG TGGCGCGGTA TTATCCCGTA
4451 TTGACGCCGG GCAAGAGCAA CTCGGTCGCC GCATACACTA TTCTCAGAAT
4501 GACTTGGTTG AGTACTCACC AGTCACAGAA AAGCATCTTA CGGATGGCAT
4551 GACAGTAAGA GAATTATGCA GTGCTGCCAT AACCATGAGT GATAACACTG
4601 CGGCCAACTT ACTTCTGACA ACGATCGGAG GACCGAAGGA GCTAACCGCT
4651 TTTTTGCACA ACATGGGGGA TCATGTAACT CGCCTTGATC GTTGGGAACC
4701 GGAGCTGAAT GAAGCCATAC CAAACGACGA GCGTGACACC ACGATGCCTG
4751 TAGCAATGGC AACAACGTTG CGCAAACTAT TAACTGGCGA ACTACTTACT
4801 CTAGCTTCCC GGCAACAATT AATAGACTGG ATGGAGGCGG ATAAAGTTGC
4851 AGGACCACTT CTGCGCTCGG CCCTTCCGGC TGGCTGGTTT ATTGCTGATA
4901 AATCTGGAGC CGGTGAGCGT GGGTCTCGCG GTATCATTGC AGCACTGGGG
4951 CCAGATGGTA AGCCCTCCCG TATCGTAGTT ATCTACACGA CGGGGAGTCA
5001 GGCAACTATG GATGAACGAA ATAGACAGAT CGCTGAGATA GGTGCCTCAC
5051 TGATTAAGCA TTGGTAACTG TCAGACCAAG TTTACTCATA TATACTTTAG
5101 ATTGATTTAA AACTTCATTT TTAATTTAAA AGGATCTAGG TGAAGATCCT
5151 TTTTGATAAT CTCATGACCA AAATCCCTTA ACGTGAGTTT TCGTTCCACT
5201 GAGCGTCAGA CCCCGTAGAA AAGATCAAAG GATCTTCTTG AGATCCTTTT
5251 TTTCTGCGCG TAATCTGCTG CTTGCAAACA AAAAAACCAC CGCTACCAGC
5301 GGTGGTTTGT TTGCCGGATC AAGAGCTACC AACTCTTTTT CCGAAGGTAA
5351 CTGGCTTCAG CAGAGCGCAG ATACCAAATA CTGTTCTTCT AGTGTAGCCG
5401 TAGTTAGGCC ACCACTTCAA GAACTCTGTA GCACCGCCTA CATACCTCGC
5451 TCTGCTAATC CTGTTACCAG TGGCTGCTGC CAGTGGCGAT AAGTCGTGTC
5501 TTACCGGGTT GGACTCAAGA CGATAGTTAC CGGATAAGGC GCAGCGGTCG
5551 GGCTGAACGG GGGGTTCGTG CACACAGCCC AGCTTGGAGC GAACGACCTA
5601 CACCGAACTG AGATACCTAC AGCGTGAGCT ATGAGAAAGC GCCACGCTTC
5651 CCGAAGGGAG AAAGGCGGAC AGGTATCCGG TAAGCGGCAG GGTCGGAACA
5701 GGAGAGCGCA CGAGGGAGCT TCCAGGGGGA AACGCCTGGT ATCTTTATAG
5751 TCCTGTCGGG TTTCGCCACC TCTGACTTGA GCGTCGATTT TTGTGATGCT
5801 CGTCAGGGGG GCGGAGCCTA TGGAAAAACG CCAGCAACGC GGCCTTTTTA
5851 CGGTTCCTGG CCTTTTGCTG GCCTTTTGCT CACATGTTCT TTCCTGCGTT
5901 ATCCCCTGAT TCTGTGGATA ACCGTATTAC CGCCTTTGAG TGAGCTGATA
5951 CCGCTCGCCG CAGCCGAACG ACCGAGCGCA GCGAGTCAGT GAGCGAGGAA
6001 GCGGAAGAGC GCCCAATACG CAAACCGCCT CTCCCCGCGC GTTGGCCGAT
6051 TCATTAATG
```

In one embodiment, a scAAV vector has a mCMV promoter, a sNRP-1 Poly A signal, and hIDUA^{op}, which is codon optimized and has a sequence at least 90% identical to SEQ DD NO:5 (ptrs-mCMV-hIDUA^{op}), e.g., at least 91%, 92%, 93%, 94%, 95%, 96%, 97%, 98%, 99%, 99.5%, or 99.9% identical to SEQ ID NO:5.

```
SEQ ID NO: 5
      1 CAGCAGCTGC GCGCTCGCTC GCTCACTGAG GCCGCCCGGG CAAAGCCCGG
    51 GCGTCGGGCG ACCTTTGGTC GCCCGGCCTC AGTGAGCGAG CGAGCGCGCA
  101 GAGAGGGAGT GGGGTTATTT CGTATTTTAT TTAGATCTGA TGGTCAGGGA
  151 TTGCCCGGGC TGGGGGGTCC GCGCGGAACA GGGACCTCCA AATATGGGAC
  201 TGGATCGGAG AAGGGGCCGG GCCTAGCCCA GTAGTCCAAG GCCCGAACTC
  251 GGTAGGAGCC TGACACGGCT CCCGTGTCGG GGGAGAATAC AAACAGGTTG
  301 AACGTTGAGG GTTTCCTAGA TACCGGGGTG TATGCTTTGC CATCTTGTGA
  351 GAACTGTATT TCATAAGTCC AGAGGCATTT AGAGCCCACA TGCTCGTCGC
  401 TCCAAACGAG GACAAGTTGT CCTTGAGTCA AAGGAAGAGC TCGCAAGCGA
  451 GTGACCTGGC CAGGGGGTTT CTCAGGTCGG GCACATACGT GTACCAAGAG
  501 AAGACTGGGG AGCCTGAGAG CGGGTCTAAG GGTGAGGGGG CCCCCAGCGG
  551 GGAGAGGTCT TGGCGCTGCC GCTACTGGGT CCTCAGCGGC CCTCATTCGC
  601 CGGAATTGTT CCGCGGTCGG GAAAACAGGC CGTCCAAGTC TTCTCCACTC
  651 GCCATCCGGA GAACAGAGTC CGTTGTCGAG GTAGCGAGTG ACGTAAACGA
  701 GACCGGGACC AGGGGGCACT CCGCGGAGCC TCAGCGTCAC CGCGACTGAT
  751 CTGTTCGGAT GGGCCCTAGT GTCGTCACTG GCGTAGATCA GAACTGCAGC
  801 TCGCCAAGCA TCAGCAGGTC CTTGTGGCCT GTGCGCGGAG GCGAGGACCC
  851 CTACGGTGTG GTTGCTATCC AATACAGTCC CTGCTTGAGA TACCTCCGCC
  901 CACAGCTGTT CCTCGTCGAG AAGTGCCAGA AGTCCCATGG CAGTAAGCAC
  951 AGGTTTTCTC AACAACTGGA CGTGAGGAGG TCGTGTGTTA TTGACCTGGA
1001 ACCGCGCTGT GAGGGTTCGC TGTGCAAAGG GATGCGGGTG GTATGAAAGG
1051 AAAGCGTTAT CGTTTGAAAG CAAGGCATAC GGAAAAGCGG ATGTCGTGTT
1101 TGCCAGGAGC AAGTTTTGAT GTTGCGCGAT CACTTTGACT ACCATTGCTG
1151 CATATGTCAC ATCCGCCCGC CAGGGTTGCG GAAGTGACCA ACCCACAAGA
1201 GGGTCGGCCT CATCATTATA GATAGGGGTG TCGGCAAATT TAGGAAAGAG
1251 TTGTCTAATT TGTTGTGCCA CGACCTTCTC CTGCTCCAGG ATAGAGATTG
1301 AGGACCTCGC ACCTTTTCGA TGCAGAGAAA TATAATCGAG GCGGACGCCC
1351 GCTTCTCCTG TAAAAAAGTT CGTACCGTCA TGACAGTGTC GCAACAGCCC
1401 CCAAGAAAGC GGGGACCGAG GCGGTGTGTG GAAGGAGTCC CCTGGGCCCC
1451 CCAGGCGGAG TGCCGGACTG GCTGCTCTCA GTCCTTCAGA ACACGCGTCA
1501 TAGTAGTTGA GAAAGCCCTG CATCGTCATA GAAACATTGT CAAAATCGTG
1551 GTGATCAGGC TCATTCCACG TCTCAAAGTT CCACTTACTT ACGTGCGCAA
1601 GCCCGTAGCG CCCTATATAC CTCCGGGCAA GTGATGACAC GAGGTCTTTC
1651 CACTCAAAAA CTTGTTGCTT ATCCTCGAAA TCAGTGAAGT GTCCGCTAGC
1701 TGACCCCATC AACTCGAAAC CGGGAAGCAG CTGATTCTCT CGCAGGAGGT
1751 CAAGGTATCC GTCCAAGTGA GTGAAATTGT AAGAGAGTCC CCTTCCTGTG
1801 CTGCCCCGTG TCGTAACCAA CTCAAGGAGC CAATGGGTTC GAACCTGTTT
1851 AATTCCGCGA TGAGGTACGG CACCGACATA GGCCAGGTTC AGTTGCTGAT
1901 CCCAAGACAA CACATACTGA TCCGCTTGAG AGTGTGGCAA TGGAGGACAG
1951 AACCCCGTTG ACCTCCAAAA CCTCCGAAGC GGCCACAGTG CTCGTGCTGC
2001 ATCAACATGC ACGAGGTGGG GTGCTTCTGC TGGAGCGACA GGGGGTGCGG
2051 CCAACAGGGA CGCCAAAAGA GCCAACAACG CAGCCCGTGG CCTCAGAGGT
2101 CGCATGGCGA CCGGTCGAGC GGTTCACTAA ACGAGCTCTG CTTATATAGA
2151 CCTCCCACCG TACACGCCTA CCGCCCATTT GCGTCAATGG GGCGGAGTTG
2201 TTACGACATT TTGGAAAGTC CCGTTGATTT TGGTGCCAAA ACAAACTCCC
2251 ATTGACGTCA ATGGGGTGGA GACTTGGAAA TCCCCGTGAG TCCGGGGGTC
2301 GTTGGGCGGT CAGCCAGGCG GGCCATTTAC CGTAAGTTAT GTAACGAAAG
2351 CTTCCTAGGA ACCCCTAGTG ATGGAGTTGG CCACTCCCTC TCTGCGCGCT
2401 CGCTCGCTCA CTGAGGCCGG GCGACCAAAG GTCGCCCGAC GCCCGGGCTT
2451 TGCCCGGGCG GCCTCAGTGA GCGAGCGAGC GCGCAGCTGG CGTAATAGCG
2501 AAGAGGCCCG CACCGATCGC CCTTCCCAAC AGTTGCGCAG CCTGAATGGC
2551 GAATGGCGAT TCCGTTGCAA TGGCTGGCGG TAATATTGTT CTGGATATTA
2601 CCAGCAAGGC CGATAGTTTG AGTTCTTCTA CTCAGGCAAG TGATGTTATT
2651 ACTAATCAAA GAAGTATTGC GACAACGGTT AATTTGCGTG ATGGACAGAC
2701 TCTTTTACTC GGTGGCCTCA CTGATTATAA AAACACTTCT CAGGATTCTG
2751 GCGTACCGTT CCTGTCTAAA ATCCCTTTAA TCGGCCTCCT GTTTAGCTCC
2801 CGCTCTGATT CTAACGAGGA AAGCACGTTA TACGTGCTCG TCAAAGCAAC
2851 CATAGTACGC GCCCTGTAGC GGCGCATTAA GCGCGGGGGG TGTGGTGGTT
2901 ACGCGCAGCG TGACCGCTAC ACTTGCCAGC GCCCTAGCGC CCGCTCCTTT
2951 CGCTTTCTTC CCTTCCTTTC TCGCCACGTT CGCCGGCTTT CCCCGTCAAG
3001 CTCTAAATCG GGGGCTCCCT TTAGGGTTCC GATTTAGTGC TTTACGGCAC
3051 CTCGACCCCA AAAAACTTGA TTAGGGTGAT GGTTCACGTA GTGGGCCATC
3101 GCCCTGATAG ACGGTTTTTC GCCCTTTGAC GTTGGAGTCC ACGTTCTTTA
3151 ATAGTGGACT CTTGTTCCAA ACTGGAACAA CACTCAACCC TATCTCGGTC
3201 TATTCTTTTG ATTTATAAGG GATTTTGCCG ATTTCGGCCT ATTGGTTAAA
3251 AAATGAGCTG ATTTAACAAA AATTTAACGC GAATTTTAAC AAAATATTAA
3301 CGCTTACAAT TTAAATATTT GCTTATACAA TCTTCCTGTT TTTGGGGCTT
3351 TTCTGATTAT CAACCGGGGT ACATATGATT GACATGCTAG TTTTACGATT
3401 ACCGTTCATC GATTCTCTTG TTTGCTCCAG ACTCTCAGGC AATGACCTGA
3451 TAGCCTTTGT AGAGACCTCT CAAAAATAGC TACCCTCTCC GGCATGAATT
3501 TATCAGCTAG AACGGTTGAA TATCATATTG ATGGTGATTT GACTGTCTCC
3551 GGCCTTTCTC ACCCGTTTGA ATCTTTACCT ACACATTACT CAGGCATTGC
3601 ATTTAAAATA TATGAGGGTT CTAAAAATTT TTATCCTTGC GTTGAAATAA
3651 AGGCTTCTCC CGCAAAAGTA TTACAGGGTC ATAATGTTTT TGGTACAACC
3701 GATTTAGCTT TATGCTCTGA GGCTTTATTG CTTAATTTTG CTAATTCTTT
3751 GCCTTGCCTG TATGATTTAT TGGATGTTGG AATCGCCTGA TGCGGTATTT
3801 TCTCCTTACG CATCTGTGCG GTATTTCACA CCGCATATGG TGCACTCTCA
3851 GTACAATCTG CTCTGATGCC GCATAGTTAA GCCAGCCCCG ACACCCGCCA
3901 ACACCCGCTG ACGCGCCCTG ACGGGCTTGT CTGCTCCCGG CATCCGCTTA
3951 CAGACAAGCT GTGACCGTCT CCGGGAGCTG CATGTGTCAG AGGTTTTCAC
4001 CGTCATCACC GAAACGCGCG AGACGAAAGG GCCTCGTGAT ACGCCTATTT
4051 TTATAGGTTA ATGTCATGAT AATAATGGTT TCTTAGACGT CAGGTGGCAC
4101 TTTTCGGGGA AATGTGCGCG GAACCCCTAT TTGTTTATTT TTCTAAATAC
4151 ATTCAAATAT GTATCCGCTC ATGAGACAAT AACCCTGATA AATGCTTCAA
4201 TAATATTGAA AAAGGAAGAG TATGAGTATT CAACATTTCC GTGTCGCCCT
4251 TATTCCCTTT TTTGCGGCAT TTTGCCTTCC TGTTTTTGCT CACCCAGAAA
4301 CGCTGGTGAA AGTAAAAGAT GCTGAAGATC AGTTGGGTGC ACGAGTGGGT
4351 TACATCGAAC TGGATCTCAA CAGCGGTAAG ATCCTTGAGA GTTTTCGCCC
4401 CGAAGAACGT TTTCCAATGA TGAGCACTTT TAAAGTTCTG CTATGTGGCG
4451 CGGTATTATC CCGTATTGAC GCCGGGCAAG AGCAACTCGG TCGCCGCATA
4501 CACTATTCTC AGAATGACTT GGTTGAGTAC TCACCAGTCA CAGAAAAGCA
4551 TCTTACGGAT GGCATGACAG TAAGAGAATT ATGCAGTGCT GCCATAACCA
4601 TGAGTGATAA CACTGCGGCC AACTTACTTC TGACAACGAT CGGAGGACCG
4651 AAGGAGCTAA CCGCTTTTTT GCACAACATG GGGGATCATG TAACTCGCCT
4701 TGATCGTTGG GAACCGGAGC TGAATGAAGC CATACCAAAC GACGAGCGTG
4751 ACACCACGAT GCCTGTAGCA ATGGCAACAA CGTTGCGCAA ACTATTAACT
4801 GGCGAACTAC TTACTCTAGC TTCCCGGCAA CAATTAATAG ACTGGATGGA
4851 GGCGGATAAA GTTGCAGGAC CACTTCTGCG CTCGGCCCTT CCGGCTGGCT
4901 GGTTTATTGC TGATAAATCT GGAGCCGGTG AGCGTGGGTC TCGCGGTATC
4951 ATTGCAGCAC TGGGGCCAGA TGGTAAGCCC TCCCGTATCG TAGTTATCTA
5001 CACGACGGGG AGTCAGGCAA CTATGGATGA ACGAAATAGA CAGATCGCTG
5051 AGATAGGTGC CTCACTGATT AAGCATTGGT AACTGTCAGA CCAAGTTTAC
5101 TCATATATAC TTTAGATTGA TTTAAAACTT CATTTTTAAT TTAAAAGGAT
5151 CTAGGTGAAG ATCCTTTTTG ATAATCTCAT GACCAAAATC CCTTAACGTG
5201 AGTTTTCGTT CCACTGAGCG TCAGACCCCG TAGAAAAGAT CAAAGGATCT
5251 TCTTGAGATC CTTTTTTTCT GCGCGTAATC TGCTGCTTGC AAACAAAAAA
5301 ACCACCGCTA CCAGCGGTGG TTTGTTTGCC GGATCAAGAG CTACCAACTC
5351 TTTTTCCGAA GGTAACTGGC TTCAGCAGAG CGCAGATACC AAATACTGTT
5401 CTTCTAGTGT AGCCGTAGTT AGGCCACCAC TTCAAGAACT CTGTAGCACC
5451 GCCTACATAC CTCGCTCTGC TAATCCTGTT ACCAGTGGCT GCTGCCAGTG
5501 GCGATAAGTC GTGTCTTACC GGGTTGGACT CAAGACGATA GTTACCGGAT
5551 AAGGCGCAGC GGTCGGGCTG AACGGGGGGT TCGTGCACAC AGCCCAGCTT
5601 GGAGCGAACG ACCTACACCG AACTGAGATA CCTACAGCGT GAGCTATGAG
5651 AAAGCGCCAC GCTTCCCGAA GGGAGAAAGG CGGACAGGTA TCCGGTAAGC
5701 GGCAGGGTCG GAACAGGAGA GCGCACGAGG GAGCTTCCAG GGGGAAACGC
5751 CTGGTATCTT TATAGTCCTG TCGGGTTTCG CCACCTCTGA CTTGAGCGTC
5801 GATTTTTGTG ATGCTCGTCA GGGGGGCGGA GCCTATGGAA AAACGCCAGC
5851 AACGCGGCCT TTTTACGGTT CCTGGCCTTT TGCTGGCCTT TTGCTCACAT
5901 GTTCTTTCCT GCGTTATCCC CTGATTCTGT GGATAACCGT ATTACCGCCT
5951 TTGAGTGAGC TGATACCGCT CGCCGCAGCC GAACGACCGA GCGCAGCGAG
6001 TCAGTGAGCG AGGAAGCGGA AGAGCGCCCA ATACGCAAAC CGCCTCTCCC
6051 CGCGCGTTGG CCGATTCATT AATG
```

In one embodiment, a scAAV vector has a mCMV promoter, a sNRP-1 Poly A signal, and hIDUA, which has a sequence at least 90% identical to SEQ ID NO:6 (ptrs-mCMV-hIDUA), e.g., at least 91%, 92%, 93%, 94%, 95%, 96%, 97%, 98%, 99%, 99.5%, or 99.9% identical to SEQ ID NO:6.

```
SEQ ID NO: 6
      1 CAGCAGCTGC GCGCTCGCTC GCTCACTGAG GCCGCCCGGG CAAAGCCCGG
    51 GCGTCGGGCG ACCTTTGGTC GCCCGGCCTC AGTGAGCGAG CGAGCGCGCA
  101 GAGAGGGAGT GGGGTTATTT CGTATTTTAT TTAGATCTGA TGGTCATGGA
  151 TTGCCCGGGG ATGGGGGCCC TCTTGGCACA GGGACCTCCA GGTACGGCAC
  201 AGGGTCCGAG AAGGGGCCTG GTCGGGCCCA GTAGTCCAGG GCTCGAACTC
  251 GGTAGGAGCC AGAGACAGCA CCTGTGTCTG GGCTGAACAC AAAGAGGTTG
  301 AAGGTCGATG GCTTCCTGCT GACCGGGGTG TACGCCTTAC CGTCCTGAGA
  351 GAACTGGATC TCGTATGTCC ACAGGCACTT GGAGCCCACG TGTTCATCCG
  401 ACCAGACCAG AACCAGCTGC CCTTGGGTCA GGGGCAGGGC GCGGAGCCGC
  451 GTGACCTGCC CGGGCGGCTT CTCGGGGCGC GCACACACGT GCACCAGCAA
  501 AAGCGACGGC AGCCGCAGCG CGGGGCGCAG GGTCAGGGGG CCGCCGGCGG
  551 GTAAGGGGCG GGGCGCCGCG GCCACCGGGT CCTCAGCCGC GCGCATGCGC
  601 CGGAACTGCT CTGCCGTGGG GAAGACGGGC CGGCCCAGGC GCCGCCACTC
  651 GCCGTCGGGG CTGCAGAGCC CGTTGTCCAG GTAGCGCGTG ACGTAGACCA
  701 GGCCCGGGCC GGGGGGCACC CCGCGCAGCC GCAGGGTCAC CGCGACGCTG
  751 CGGTTGGGGT GGGCGCGGGT GTCGTCGCTC GCGTAGATCA GCACCGCGGC
  801 GCGCCAGGCG TCGGCCGGGC CCTGGGGGCG GTGGGCGCTG GCCAGGACGC
  851 CCACCGTGTG GTTGCTGTCC AGGACGGTCC CGGCCTGCGA CACTTCGGCC
  901 CAGAGCTGCT CCTCATCCAG CAGCGCCAGC AGCCCCATGG CCGTGAGCAC
  951 CGGCTTGCGC AACAGCTGCA CGTGCGGCGG GCGGGTGTTG TTGACCTGGA
1001 AGCGCGCGGT GAGCGTGCGC TGCGCGAAGG GGTGCGGGTG GTAGCTCAGG
1051 AAGGCATTGT CGTTGCTCAG GAGCGCGTAG GGGAAGGCGG AGGTGGTGTT
1101 GGCCAGTAGC AGGTTCTGAT GCTGCGCGAT GACCTTCACC ACCATGGCCG
1151 CGTAGGTCAC GTCCGCCCTC CACGGCTGTG GCAGGGACCA GCCCACCAGC
1201 GGGTCCGCCT CGTCGTTGTA AATGGGGGTG TCCGCGAACT TGGGGAAGAG
1251 CTGCCGGATC TGCTGCGCGA CGACCTTCTC CTGCTCCAGG ATGGAGATGG
1301 AGCTGCGCGC ACCCTTCCTG TGGAGGGAGA TGTAGTCCAG CCGCACGCCC
1351 GCCTCCCCAG TGAAGAAGTT GGTACCGTCG TGGCAGTGGC GCAGGAGGCC
1401 CCAGCTCAGC GGGGATCGCG GTGGGGTGTG GAAGGAGTCG CCGGGGCCTC
1451 CCAGCCGCAG GGCGGGGCTG GCGGCGCGCA GACCCTCCGA GCAGGCATCG
1501 TAGTAGTTCA GGAAGCCTTG CATGGTCATG GAGACGTTGT CAAAGTCGTG
1551 GTGGTCTGGC TCATTCCACG TCTCGAAGTT CCACTTGGAA ACATGCGCCA.
1601 GTCCGTACCT ACCGATGTAT CTCCTGGCCA GGCTGGAGAC CAAGTCCTTC
1651 CACTCAAACA CCTGCTGCTT GTCCTCAAAG TCAGTGAAGT GGCCCGAGGC
1701 GCTGCCCATC AGCTCAAACC CTGGGAGGAG CTGGTTCTCC CTGAGAAGGT
1751 CCAGGTACCC GTCCAGGTGG GTGAAGTTGT AGCTCAGGCC CCGTCCAGTG
1801 GACCCCCTGG TGGTGACAAG CTCCAGCAGC CAGTGGGTCC GGACCTGCTT
1851 GATGCCGCGG TGAGGGACGG CGCCCACATA GGCGAGGTTG AGCTGCTGGT
1901 CCCAGCTGAG GACGTACTGG TCAGCCTGGC TGTGTGGCAG CGGGGGGCAG
1951 AAGCCTGTGC TCCTCCAGAA GCGCCGCAGG GGCCACAGCG CGCGGGCCGC
2001 GTCCACATGC ACCAGGTGCG GGGCCTCGGC CGGGGCCACC GGGGGCGCGG
2051 CCAGGAGCGA GGCCAGGAGC GCCAGCAGCG CGGCGCGGGG GCGCAGGGGA
2101 CGCATGGCGA CCGGTCGAGC GGTTCACTAA ACGAGCTCTG CTTATATAGA
2151 CCTCCCACCG TACACGCCTA CCGCCCATTT GCGTCAATGG GGCGGAGTTG
2201 TTACGACATT TTGGAAAGTC CCGTTGATTT TGGTGCCAAA ACAAACTCCC
2251 ATTGACGTCA ATGGGGTGGA GACTTGGAAA TCCCCGTGAG TCCGGGGGTC
2301 GTTGGGGGGT CAGCCAGGCG GGCCATTTAC CGTAAGTTAT GTAACGAAAG
2351 CTTCCTAGGA ACCCCTAGTG ATGGAGTTGG CCACTCCCTC TCTGCGCGCT
2401 CGCTCGCTCA CTGAGGCCGG GCGACCAAAG GTCGCCCGAC GCCCGGGCTT
2451 TGCCCGGGCG GCCTCAGTGA GCGAGCGAGC GCGCAGCTGG CGTAATAGCG
2501 AAGAGGCCCG CACCGATCGC CCTTCCCAAC AGTTGCGCAG CCTGAATGGC
2551 GAATGGCGAT TCCGTTGCAA TGGCTGGCGG TAATATTGTT CTGGATATTA
2601 CCAGCAAGGC CGATAGTTTG AGTTCTTCTA CTCAGGCAAG TGATGTTATT
2651 ACTAATCAAA GAAGTATTGC GACAACGGTT AATTTGCGTG ATGGACAGAC
2701 TCTTTTACTC GGTGGCCTCA CTGATTATAA AAACACTTCT CAGGATTCTG
2751 GCGTACCGTT CCTGTCTAAA ATCCCTTTAA TCGGCCTCCT GTTTAGCTCC
2801 CGCTCTGATT CTAACGAGGA AAGCACGTTA TACGTGCTCG TCAAAGCAAC
2851 CATAGTACGC GCCCTGTAGC GGCGCATTAA GCGCGGGGGG TGTGGTGGTT
2901 ACGCGCAGCG TGACCGCTAC ACTTGCCAGC GCCCTAGCGC CCGCTCCTTT
2951 CGCTTTCTTC CCTTCCTTTC TCGCCACGTT CGCCGGCTTT CCCCGTCAAG
3001 CTCTAAATCG GGGGCTCCCT TTAGGGTTCC GATTTAGTGC TTTACGGCAC
3051 CTCGACCCCA AAAAACTTGA TTAGGGTGAT GGTTCACGTA GTGGGCCATC
3101 GCCCTGATAG ACGGTTTTTC GCCCTTTGAC GTTGGAGTCC ACGTTCTTTA
3151 ATAGTGGACT CTTGTTCCAA ACTGGAACAA CACTCAACCC TATCTCGGTC
3201 TATTCTTTTG ATTTATAAGG GATTTTGCCG ATTTCGGCCT ATTGGTTAAA
3251 AAATGAGCTG ATTTAACAAA AATTTAACGC GAATTTTAAC AAAATATTAA
3301 CGCTTACAAT TTAAATATTT GCTTATACAA TCTTCCTGTT TTTGGGGCTT
3351 TTCTGATTAT CAACCGGGGT ACATATGATT GACATGCTAG TTTTACGATT
3401 ACCGTTCATC GATTCTCTTG TTTGCTCCAG ACTCTCAGGC AATGACCTGA
3451 TAGCCTTTGT AGAGACCTCT CAAAAATAGC TACCCTCTCC GGCATGAATT
3501 TATCAGCTAG AACGGTTGAA TATCATATTG ATGGTGATTT GACTGTCTCC
3551 GGCCTTTCTC ACCCGTTTGA ATCTTTACCT ACACATTACT CAGGCATTGC
3601 ATTTAAAATA TATGAGGGTT CTAAAAATTT TTATCCTTGC GTTGAAATAA
3651 AGGCTTCTCC CGCAAAAGTA TTACAGGGTC ATAATGTTTT TGGTACAACC
3701 GATTTAGCTT TATGCTCTGA GGCTTTATTG CTTAATTTTG CTAATTCTTT
3751 GCCTTGCCTG TATGATTTAT TGGATGTTGG AATCGCCTGA TGCGGTATTT
3801 TCTCCTTACG CATCTGTGCG GTATTTCACA CCGCATATGG TGCACTCTCA
3851 GTACAATCTG CTCTGATGCC GCATAGTTAA GCCAGCCCCG ACACCCGCCA
3901 ACACCCGCTG ACGCGCCCTG ACGGGCTTGT CTGCTCCCGG CATCCGCTTA
3951 CAGACAAGCT GTGACCGTCT CCGGGAGCTG CATGTGTCAG AGGTTTTCAC
4001 CGTCATCACC GAAACGCGCG AGACGAAAGG GCCTCGTGAT ACGCCTATTT
4051 TTATAGGTTA ATGTCATGAT AATAATGGTT TCTTAGACGT CAGGTGGCAC
4101 TTTTCGGGGA AATGTGCGCG GAACCCCTAT TTGTTTATTT TTCTAAATAC
4151 ATTCAAATAT GTATCCGCTC ATGAGACAAT AACCCTGATA AATGCTTCAA
4201 TAATATTGAA AAAGGAAGAG TATGAGTATT CAACATTTCC GTGTCGCCCT
4251 TATTCCCTTT TTTGCGGCAT TTTGCCTTCC TGTTTTTGCT CACCCAGAAA
4301 CGCTGGTGAA AGTAAAAGAT GCTGAAGATC AGTTGGGTGC ACGAGTGGGT
4351 TACATCGAAC TGGATCTCAA CAGCGGTAAG ATCCTTGAGA GTTTTCGCCC
4401 CGAAGAACGT TTTCCAATGA TGAGCACTTT TAAAGTTCTG CTATGTGGCG
4451 CGGTATTATC CCGTATTGAC GCCGGGCAAG AGCAACTCGG TCGCCGCATA
4501 CACTATTCTC AGAATGACTT GGTTGAGTAC TCACCAGTCA CAGAAAAGCA
4551 TCTTACGGAT GGCATGACAG TAAGAGAATT ATGCAGTGCT GCCATAACCA
4601 TGAGTGATAA CACTGCGGCC AACTTACTTC TGACAACGAT CGGAGGACCG
4651 AAGGAGCTAA CCGCTTTTTT GCACAACATG GGGGATCATG TAACTCGCCT
4701 TGATCGTTGG GAACCGGAGC TGAATGAAGC CATACCAAAC GACGAGCGTG
4751 ACACCACGAT GCCTGTAGCA ATGGCAACAA CGTTGCGCAA ACTATTAACT
4801 GGCGAACTAC TTACTCTAGC TTCCCGGCAA CAATTAATAG ACTGGATGGA
4851 GGCGGATAAA GTTGCAGGAC CACTTCTGCG CTCGGCCCTT CCGGCTGGCT
4901 GGTTTATTGC TGATAAATCT GGAGCCGGTG AGCGTGGGTC TCGCGGTATC
4951 ATTGCAGCAC TGGGGCCAGA TGGTAAGCCC TCCCGTATCG TAGTTATCTA
5001 CACGACGGGG AGTCAGGCAA CTATGGATGA ACGAAATAGA CAGATCGCTG
5051 AGATAGGTGC CTCACTGATT AAGCATTGGT AACTGTCAGA CCAAGTTTAC
5101 TCATATATAC TTTAGATTGA TTTAAAACTT CATTTTTAAT TTAAAAGGAT
5151 CTAGGTGAAG ATCCTTTTTG ATAATCTCAT GACCAAAATC CCTTAACGTG
5201 AGTTTTCGTT CCACTGAGCG TCAGACCCCG TAGAAAAGAT CAAAGGATCT
5251 TCTTGAGATC CTTTTTTTCT GCGCGTAATC TGCTGCTTGC AAACAAAAAA
5301 ACCACCGCTA CCAGCGGTGG TTTGTTTGCC GGATCAAGAG CTACCAACTC
5351 TTTTTCCGAA GGTAACTGGC TTCAGCAGAG CGCAGATACC AAATACTGTT
5401 CTTCTAGTGT AGCCGTAGTT AGGCCACCAC TTCAAGAACT CTGTAGCACC
5451 GCCTACATAC CTCGCTCTGC TAATCCTGTT ACCAGTGGCT GCTGCCAGTG
5501 GCGATAAGTC GTGTCTTACC GGGTTGGACT CAAGACGATA GTTACCGGAT
5551 AAGGCGCAGC GGTCGGGCTG AACGGGGGGT TCGTGCACAC AGCCCAGCTT
5601 GGAGCGAACG ACCTACACCG AACTGAGATA CCTACAGCGT GAGCTATGAG
5651 AAAGCGCCAC GCTTCCCGAA GGGAGAAAGG CGGACAGGTA TCCGGTAAGC
5701 GGCAGGGTCG GAACAGGAGA GCGCACGAGG GAGCTTCCAG GGGGAAACGC
5751 CTGGTATCTT TATAGTCCTG TCGGGTTTCG CCACCTCTGA CTTGAGCGTC
5801 GATTTTTGTG ATGCTCGTCA GGGGGGCGGA GCCTATGGAA AAACGCCAGC
5851 AACGCGGCCT TTTTACGGTT CCTGGCCTTT TGCTGGCCTT TTGCTCACAT
5901 GTTCTTTCCT GCGTTATCCC CTGATTCTGT GGATAACCGT ATTACCGCCT
5951 TTGAGTGAGC TGATACCGCT CGCCGCAGCC GAACGACCGA GCGCAGCGAG
6001 TCAGTGAGCG AGGAAGCGGA AGAGCGCCCA ATACGCAAAC CGCCTCTCCC
6051 CGCGCGTTGG CCGATTCATT AATG
```

The viral vector may further comprise a promoter operably linked to the IDUA nucleic acid. In some embodiments, the promoter may be a constitutive promoter, e.g., a CMV promoter. In other embodiments, the promoter may be a tissue-specific or preferred promoter. In some embodiments, the preferred promoter is as cytomegalovirus (CMV) promoter, optionally the preferred promoter is a truncated miniature CMV promoter (mCMV), further optionally the preferred promoter is a 228 bp mCMV having a sequence identical to SEQ ID NO:7. In some embodiments, the nucleic acid is operably linked to a polyadenylation signal, optionally wherein the polyadenylation signal is less than 25 nucleotides in length, optionally wherein the polyadenylation signal is 16 nucleotides in length having a sequence identical to SEQ ID NO:8. In some embodiments, the polyadenylation signal is a sNRP-1 polyadenylation signal. The invention further provides a cell in vitro comprising the AAV vector genome of the invention stably incorporated into the genome of the cell. The invention further provides a recombinant parvovirus particle (e.g., a recombinant AAV particle) comprising the viral vector genome of the invention. Viral vectors and viral particles are discussed further below.

```
SEQ ID NO: 7
    1 CGGTTCACTA AACGAGCTCT GCTTATATAG ACCTCCCACC GTACACGCCT
  51 ACCGCCCATT TGCGTCAATG GGGCGGAGTT GTTACGACAT TTTGGAAAGT
101 CCCGTTGATT TTGGTGCCAA AACAAACTCC CATTGACGTC AATGGGGTGG
151 AGACTTGGAA ATCCCCGTGA GTCCGGGGGT CGTTGGGGGG TCAGCCAGGC
201 GGGCCATTTA CCGTAAGTTA TGTAACGA
SEQ ID NO: 8
    1 AAATAAAATA CGAAAT
```

### Methods of Producing Virus Vectors

The present invention further provides methods of producing virus vectors. In one particular embodiment, the present invention provides a method of producing a recombinant parvovirus particle, comprising providing to a cell permissive for parvovirus replication: (a) a recombinant parvovirus template comprising (i) a nucleic acid encoding IDUA, and (ii) a parvovirus ITR; (b) a polynucleotide comprising Rep and Cap coding sequences; under conditions sufficient for the replication and packaging of the recombinant parvovirus template; whereby recombinant parvovirus particles are produced in the cell. Conditions sufficient for the replication and packaging of the recombinant parvovirus template can be, e.g., the presence of AAV sequences sufficient for replication of the parvovirus template and encapsidation into parvovirus capsids (e.g., parvovirus rep sequences and parvovirus cap sequences) and helper sequences from adenovirus and/or herpesvirus. In particular embodiments, the parvovirus template comprises two parvovirus ITR sequences, which are located 5′ and 3′ to the heterologous nucleic acid sequence, although they need not be directly contiguous thereto.

In some embodiments, the recombinant parvovirus template comprises an ITR that is not resolved by Rep to make duplexed AAV vectors as described in international patent publication WO 01/92551.

The parvovirus template and parvovirus rep and cap sequences are provided under conditions such that virus vector comprising the parvovirus template packaged within the parvovirus capsid is produced in the cell. The method can further comprise the step of collecting the virus vector from the cell. The virus vector can be collected from the medium and/or by lysing the cells.

In some embodiments, an AAV particle is provided comprising an AAV vector genome.

The cell can be a cell that is permissive for parvoviral viral replication. Any suitable cell known in the art may be employed. In particular embodiments, the cell is a mammalian cell (e.g., a primate or human cell). As another option, the cell can be a trans-complementing packaging cell line that provide functions deleted from a replication-defective helper virus, e.g., 293 cells or other E1a trans-complementing cells.

The parvovirus replication and capsid sequences may be provided by any method known in the art. Current protocols typically express the parvovirus rep/cap genes on a single plasmid. The parvovirus replication and packaging sequences need not be provided together, although it may be convenient to do so. The parvovirus rep and/or cap sequences may be provided by any viral or non-viral vector. For example, the rep/cap sequences may be provided by a hybrid adenovirus or herpesvirus vector (e.g., inserted into the E1a or E3 regions of a deleted adenovirus vector). EBV vectors may also be employed to express the parvovirus cap and rep genes. One advantage of this method is that EBV vectors are episomal, yet will maintain a high copy number throughout successive cell divisions (i.e., are stably integrated into the cell as extra-chromosomal elements, designated as an “EBV based nuclear episome,” see Margolski, (1992) Curr. Top. Microbiol. Immun. 158:67).

As a further alternative, the rep/cap sequences may be stably incorporated into a cell.

Typically the parvovirus rep/cap sequences will not be flanked by the TRs, to prevent rescue and/or packaging of these sequences.

The parvovirus template can be provided to the cell using any method known in the art. For example, the template can be supplied by a non-viral (e.g., plasmid) or viral vector. In particular embodiments, the parvovirus template is supplied by a herpesvirus or adenovirus vector (e.g., inserted into the E1a or E3 regions of a deleted adenovirus). As another illustration, Palombo et al., (1998) J. Virology 72:5025, describes a baculovirus vector carrying a reporter gene flanked by the AAV TRs. EBV vectors may also be employed to deliver the template, as described above with respect to the rep/cap genes.

In another representative embodiment, the parvovirus template is provided by a replicating rAAV virus. In still other embodiments, an AAV provirus comprising the parvovirus template is stably integrated into the chromosome of the cell.

To enhance virus titers, helper virus functions (e.g., adenovirus or herpesvirus) that promote a productive parvovirus infection can be provided to the cell. Helper virus sequences necessary for parvovirus replication are known in the art. Typically, these sequences will be provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes that promote efficient parvovirus production as described by Ferrari et al., (1997) Nature Med. 3:1295, and U.S. Pat. Nos. 6,040,183 and 6,093,570.

Further, the helper virus functions may be provided by a packaging cell with the helper sequences embedded in the chromosome or maintained as a stable extrachromosomal element. Generally, the helper virus sequences cannot be packaged into AAV virions, e.g., are not flanked by ITRs.

Those skilled in the art will appreciate that it may be advantageous to provide the parvovirus replication and capsid sequences and the helper virus sequences (e.g., adenovirus sequences) on a single helper construct. This helper construct may be a non-viral or viral construct. As one nonlimiting illustration, the helper construct can be a hybrid adenovirus or hybrid herpesvirus comprising the AAV rep/cap genes.

In one particular embodiment, the parvovirus rep/cap sequences and the adenovirus helper sequences are supplied by a single adenovirus helper vector. This vector can further comprise the parvovirus template. The parvovirus rep/cap sequences and/or the parvovirus template can be inserted into a deleted region (e.g., the E1a or E3 regions) of the adenovirus.

In a further embodiment, the parvovirus rep/cap sequences and the adenovirus helper sequences are supplied by a single adenovirus helper vector. According to this embodiment, the parvovirus template can be provided as a plasmid template.

In another illustrative embodiment, the parvovirus rep/cap sequences and adenovirus helper sequences are provided by a single adenovirus helper vector, and the parvovirus template is integrated into the cell as a provirus. Alternatively, the parvovirus template is provided by an EBV vector that is maintained within the cell as an extrachromosomal element (e.g., as an EBV based nuclear episome).

In a further exemplary embodiment, the parvovirus rep/cap sequences and adenovirus helper sequences are provided by a single adenovirus helper. The parvovirus template can be provided as a separate replicating viral vector. For example, the parvovirus template can be provided by a parvovirus particle or a second recombinant adenovirus particle.

According to the foregoing methods, the hybrid adenovirus vector typically comprises the adenovirus 5′ and 3′ cis sequences sufficient for adenovirus replication and packaging (i.e., the adenovirus terminal repeats and PAC sequence). The parvovirus rep/cap sequences and, if present, the AAV template are embedded in the adenovirus backbone and are flanked by the 5′ and 3′ cis sequences, so that these sequences may be packaged into adenovirus capsids. As described above, the adenovirus helper sequences and the parvovirus rep/cap sequences are generally not flanked by ITRs so that these sequences are not packaged into the parvovirus virions.

Zhang et al., ((2001) Gene Ther. 18:704-12) describe a chimeric helper comprising both adenovirus and the AAV rep and cap genes.

Herpesvirus may also be used as a helper virus in parvovirus packaging methods. Hybrid herpesviruses encoding the parvovirus Rep protein(s) may advantageously facilitate scalable parvovirus vector production schemes. A hybrid herpes simplex virus type I (HSV-1) vector expressing the AAV-2 rep and cap genes has been described (Conway et al., (1999) Gene Ther. 6:986 and WO 00/17377.

As a further alternative, the virus vectors of the invention can be produced in insect cells using baculovirus vectors to deliver the rep/cap genes and parvovirus template as described, for example, by Urabe et al., (2002) Human Gene Ther. 13:1935-43.

Parvovirus vector stocks free of contaminating helper virus may be obtained by any method known in the art. For example, parvovirus and helper virus may be readily differentiated based on size. Parvovirus may also be separated away from helper virus based on affinity for a heparin substrate (Zolotukhin et al., (1999) Gene Therapy 6:973). Deleted replication-defective helper viruses can be used so that any contaminating helper virus is not replication competent. As a further alternative, an adenovirus helper lacking late gene expression may be employed, as only adenovirus early gene expression is required to mediate packaging of parvovirus. Adenovirus mutants defective for late gene expression are known in the art (e.g., ts100K and ts149 adenovirus mutants).

### Recombinant Virus Vectors

The virus vectors of the present invention are useful for the delivery of nucleic acids to cells in vitro, ex vivo, and in vivo. In particular, the virus vectors can be advantageously employed to deliver or transfer nucleic acids to animal, including mammalian, cells. In particular, the virus vectors of the present invention are useful for the delivery of a nucleic acid encoding IDUA to a subject.

It will be understood by those skilled in the art that the nucleic acid encoding IDUA can be operably associated with appropriate control sequences. For example, the nucleic acid can be operably associated with expression control elements, such as transcription/translation control signals, origins of replication, polyadenylation signals, internal ribosome entry sites (IRES), promoters, and/or enhancers, and the like.

Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In particular embodiments, the promoter/enhancer elements can be native to the target cell or subject to be treated. In representative embodiments, the promoters/enhancer element can be native to the IDUA nucleic acid sequence. The promoter/enhancer element is generally chosen so that it functions in the target cell(s) of interest. Further, in particular embodiments the promoter/enhancer element is a mammalian promoter/enhancer element. The promoter/enhancer element may be constitutive or inducible.

Inducible expression control elements are typically advantageous in those applications in which it is desirable to provide regulation over expression of the nucleic acid sequence. Inducible promoters/enhancer elements for gene delivery can be tissue-specific or -preferred promoter/enhancer elements, and include eye specific or preferred (including retina-specific and cornea-specific) promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In some embodiments, the promoter is 250 base pairs or less in length. In other embodiments, the promoter is a mCMV promoter. In some embodiments, the mCMV promoter is 228 nucleotides in length. In embodiments wherein the nucleic acid sequence is transcribed and then translated in the target cells, specific initiation signals are generally included for efficient translation of inserted protein coding sequences. These exogenous translational control sequences, which may include the ATG initiation codon and adjacent sequences, can be of a variety of origins, both natural and synthetic.

In some embodiments, the AAV vector genome is self-complementary.

The virus vectors of the invention can be parvovirus vectors, e.g., AAV vectors. The AAV vectors may be any AAV serotype. In some embodiments, the AAV vector is an AAV2, AAV8, or AAV9 vector. In some embodiments, the AAV vector is a chimeric AAV8/AAV9 particle.

The virus vectors according to the present invention provide a means for delivering IDUA nucleic acids into a broad range of cells, including dividing and non-dividing cells. The virus vectors can be employed to deliver the nucleic acid to a cell in vitro, e.g., to produce IDUA in vitro or for ex vivo gene therapy. The virus vectors are additionally useful in a method of delivering the nucleic acid to a subject in need thereof, e.g., to express IDUA. In this manner, IDUA can be produced in vivo in the subject. The subject can be in need of IDUA because the subject has a deficiency of functional IDUA. Further, the method can be practiced because the production of IDUA in the subject may impart some beneficial effect.

In some embodiments, the AAV vector genome is stably incorporated into the cell genome.

The virus vectors can also be used to produce IDUA in cultured cells or in a subject (e.g., using the subject as a bioreactor to produce the polypeptide or to observe the effects of the polypeptide on the subject, for example, in connection with screening methods).

The virus vectors of the present invention can be employed to deliver a nucleic acid encoding IDUA to treat and/or prevent any disease state for which it is beneficial to deliver IDUA, e.g., MPS I.

Virus vectors according to the instant invention find use in diagnostic and screening methods, whereby the IDUA nucleic acid is transiently or stably expressed in a cell culture system, in an organ or organ culture, or alternatively, a transgenic animal model.

The virus vectors of the present invention can also be used for various non-therapeutic purposes, including but not limited to use in protocols to assess gene targeting, clearance, transcription, translation, etc., as would be apparent to one skilled in the art. The virus vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

Alternatively, the virus vector may be administered to a cell ex vivo and the altered cell is administered to the subject. The virus vector comprising the IDUA nucleic acid is introduced into the cell, and the cell is administered to the subject, where the nucleic acid can be expressed.

### Subjects, Pharmaceutical Formulations, and Modes of Administration

Virus vectors according to the present invention find use in both veterinary and medical applications. Suitable subjects include both avians and mammals. The term “avian” as used herein includes, but is not limited to, chickens, ducks, geese, quail, turkeys, pheasant, parrots, parakeets, and the like. The term “mammal” as used herein includes, but is not limited to, humans, non-human primates, bovines, ovines, caprines, equines, felines, canines, lagomorphs, etc. Human subjects include neonates, infants, juveniles, and adults.

In particular embodiments, the present invention provides a pharmaceutical composition comprising a virus vector of the invention in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form.

By “pharmaceutically acceptable” it is meant a material that is not toxic or otherwise undesirable, i.e., the material may be administered to a subject without causing any undesirable biological effects.

One aspect of the present invention is a method of transferring a nucleic acid to a cell in vitro. The virus vector may be introduced into the cells at the appropriate multiplicity of infection according to standard transduction methods suitable for the particular target cells. Titers of virus vector to administer can vary, depending upon the target cell type and number, and the particular virus vector, and can be determined by those of skill in the art without undue experimentation. In representative embodiments, at least about 10³ infectious units, more preferably at least about 10⁵ infectious units are introduced to the cell.

The cell(s) into which the virus vector is introduced can be of any type. Moreover, the cell can be from any species of origin, as indicated above.

The virus vector can be introduced into cells in vitro for the purpose of administering the modified cell to a subject. In particular embodiments, the cells have been removed from a subject, the virus vector is introduced therein, and the cells are then administered back into the subject. Methods of removing cells from subject for manipulation ex vivo, followed by introduction back into the subject are known in the art (see, e.g., U.S. Pat. No. 5,399,346). Alternatively, the recombinant virus vector can be introduced into cells from a donor subject, into cultured cells, or into cells from any other suitable source, and the cells are administered to a subject in need thereof (i.e., a “recipient” subject).

Suitable cells for ex vivo gene delivery are as described above. Dosages of the cells to administer to a subject will vary upon the age, condition and species of the subject, the type of cell, the nucleic acid being expressed by the cell, the mode of administration, and the like. Typically, at least about 10² to about 10⁸ cells or at least about 10³ to about 10⁶ cells will be administered per dose in a pharmaceutically acceptable carrier. In particular embodiments, the cells transduced with the virus vector are administered to the subject in a treatment effective or prevention effective amount in combination with a pharmaceutical carrier.

In some embodiments, IDUA is delivered to a cell in vitro or ex vivo by contacting the cell with an effective amount of the AAV particle, thereby delivering IDUA to the cell. In some embodiments, the cells are then transplanted to a subject in need thereof.

A further aspect of the invention is a method of administering the virus vector to subjects. Administration of the virus vectors according to the present invention to a human subject or an animal in need thereof can be by any means known in the art. Optionally, the virus vector is delivered in a treatment effective or prevention effective dose in a pharmaceutically acceptable carrier.

Dosages of the virus vector to be administered to a subject depend upon the mode of administration, the disease or condition to be treated and/or prevented, the individual subject's condition, the particular virus vector, and the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸ transducing units, optionally about 10⁸ to about 10¹⁵ transducing units.

In particular embodiments, more than one administration (e.g., two, three, four or more administrations) may be employed to achieve the desired level of gene expression over a period of various intervals, e.g., daily, weekly, monthly, yearly, etc.

In particular embodiments, a virus vector according to the present invention is administered to the subject to treat, delay the onset of and/or prevent MPS I.

Thus, as one aspect, the invention further encompasses a method of delivering IDUA to a subject, comprising administering to the subject an effective amount of an AAV particle that expresses IDUA, thereby delivering IDUA to the subject.

In another aspect, the invention further encompasses a method of treating, delaying the onset of, and/or preventing MPS I in a subject in need thereof, comprising administering to the subject a therapeutically effective amount of an AAV particle that expresses IDUA, thereby treating or delaying the onset of MPS I in the subject.

In some embodiments, the AAV particle is administered to the subject by injection. In other embodiments, the AAV particle is administered to the subject orally or topically. In the methods of the invention, the subject may be one has been diagnosed with MPS I or is suspected of having MPS I. In certain embodiments, the subject is an infant or child, e.g., less than 18 years old, e.g., less than 18, 17, 16, 15, 14, 13, 12, 11, 10, 9, 8, 7, 6, 5, 4, 3, 2, or 1 years old.

Exemplary modes of administration include oral, rectal, transmucosal, topical, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intro-lymphatic, and the like, as well as direct tissue or organ injection (e.g., to liver, skeletal muscle, cardiac muscle, diaphragm muscle or brain). The most suitable route in any given case will depend on the nature and severity of the condition being treated and on the nature of the particular vector that is being used.

In some embodiments, the viral vector is administered directly to the CNS, e.g., the brain or the spinal cord. Any method known in the art to administer vectors directly to the CNS can be used. The vector may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes, cortex, basal ganglia, hippocampus and amygdala), limbic system, neocortex, corpus striatum, cerebrum, and inferior colliculus. The vector may also be administered to different regions of the eye such as the retina, cornea or optic nerve. The vector may be delivered into the cerebrospinal fluid (e.g., by lumbar puncture) for more disperse administration of the vector.

The delivery vector may be administered to the desired region(s) of the CNS by any route known in the art, including but not limited to, intrathecal, intracerebral, intraventricular, intranasal, intra-aural, intra-ocular (e.g., intra-vitreous, sub-retinal, anterior chamber) and peri-ocular (e.g., sub-Tenon's region) delivery or any combination thereof.

Typically, the viral vector will be administered in a liquid formulation by direct injection (e.g., stereotactic injection) to the desired region or compartment in the CNS. In some embodiments, the vector can be delivered via a reservoir and/or pump. In other embodiments, the vector may be provided by topical application to the desired region or by intra-nasal administration of an aerosol formulation. Administration to the eye or into the ear, may be by topical application of liquid droplets. As a further alternative, the vector may be administered as a solid, slow-release formulation. Controlled release of parvovirus and AAV vectors is described by international patent publication WO 01/91803.

The virus vector can be delivered to skeletal muscle by any suitable method including without limitation intravenous administration, intra-arterial administration, intraperitoneal administration, isolated limb perfusion (of leg and/or arm; see, e.g. Arruda et al., (2005) Blood 105:3458-3464), and/or direct intramuscular injection.

Injectables can be prepared in conventional forms, either as liquid solutions or suspensions, solid forms suitable for solution or suspension in liquid prior to injection, or as emulsions. Alternatively, one may administer the virus vector and/or virus capsids of the invention in a local manner, for example, in a depot or sustained-release formulation. Further, the virus vector and/or virus capsid can be delivered adhered to a surgically implantable matrix (e.g., as described in U.S. Patent Publication No. 2004-0013645).

Having described the present invention, the same will be explained in greater detail in the following examples, which are included herein for illustration purposes only, and which are not intended to be limiting to the invention.

### Example 1

### Construction and Testing of IDUA Vectors

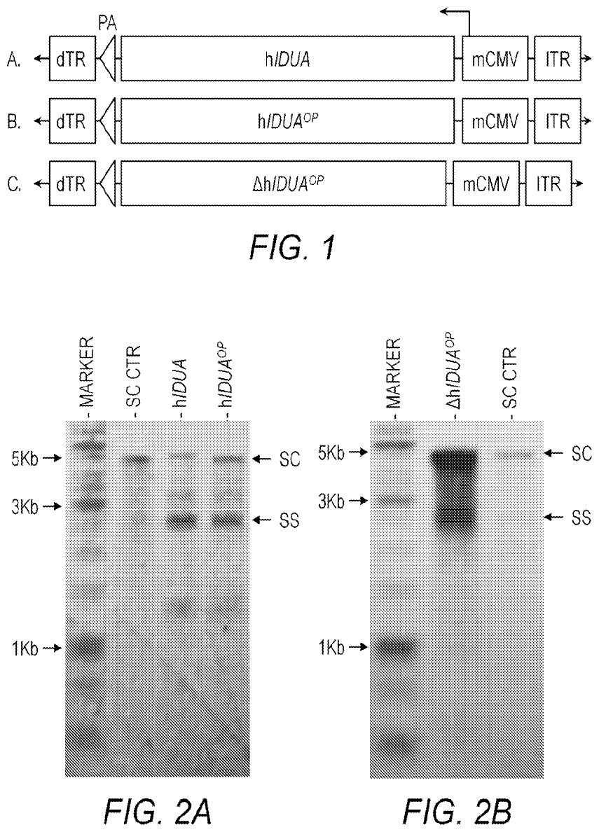

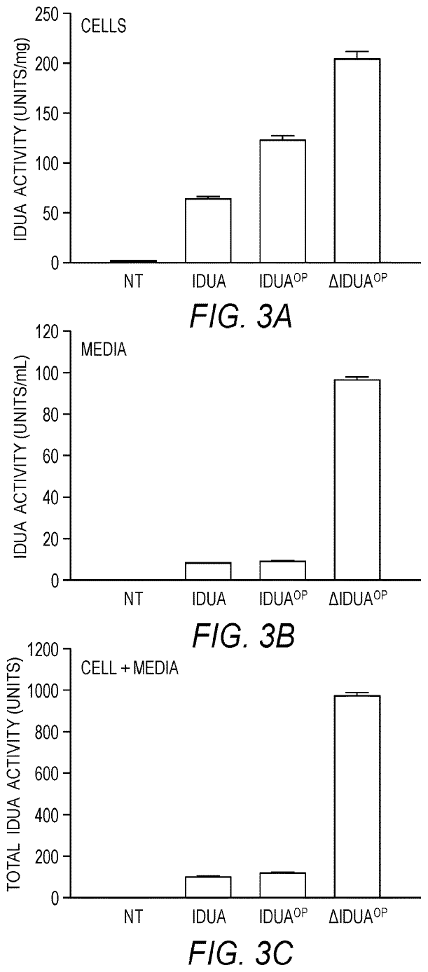

Vector Constructs: Two rAAV vector plasmids were constructed, ptrs-mCMV-hIDUA (SEQ ID NO:6) (FIG. 1, construct a) and ptrs-mCMV-hIDUA^{op} (SEQ ID NO:5) (FIG. 1, construct b), to produce scAAV vectors and enhance the transduction efficiency by codon-optimization such that effective gene therapy products for treating MPS I were developed. These vectors contain a full-length hIDUA cDNA (1962 bp) or a codon-optimized full-length hIDUA cDNA (hIDUA^{op}, 1962 bp). A 228 bp truncated miniature CMV promoter (mCMV), and a 16 bp soluble neuropilin-1 (sNRP-1) Poly A signal were used to accommodate the vector genome size limit. These resulted in the 4,850 bp vector genome, which is slightly beyond the ideal size limit for a scAAV vector (˜4,700 bp) and, as such, may not generate the desired scAAV vector. In other words, the vector may not package into a self-complementary state. In vitro testing showed that codon-optimization led to improvement in the expression and secretion of rIDUA in cell cultures (FIG. 3).

The hIDUA^{op} cDNA was miniaturized by deleting 15 nucleotides of non-conserved sequence (ΔhIDUA^{op}) near the 3′-end and constructed a scAAV vector plasmid, ptrs-mCMV-ΔhIDUA^{op} (SEQ ID NO:4) to further accommodate the vector genome for the generation of scAAV vector expressing hIDUA. The vector genome of this construct was 4,820 bp, containing ITRs, a miniaturized 1,947 bp mIDUA^{op}, a mCMV promoter and a sNRP-1 Poly A signal. FIG. 1, construct c illustrates the structure of the scAAV-mCMV-ΔhIDUA^{op} viral vector genome.

Self-Complementary AAV Viral Vector: Three separate plasmid cotransfections were performed in HEK293 cells to produce AAV9-mCMV-hIDUA, AAV9-mCMV-hIDUA^{op} or AAV9-mCMV-ΔhIDUA viral vectors to determine whether the AAV-hIDUA vector constructs would generate scAAV vectors. The purified vector products were analyzed by alkaline denaturing gel electrophoresis. The results showed that the majority (>70%) of the AAV-hIDUA and AAV-hIDUA^{op} viral vector products were ssAAV, likely because their vector genome sizes are beyond the AAV packaging capacity (FIG. 2A). However, the majority of the vector generated from the ptrs-mCMV-ΔhIDUA^{op} were scAAV (FIG. 2B) based on the mobility of the viral genome on the denaturing gel, indicating that miniaturizing hIDUA^{op} by deleting 15 bp from the 3′-end non-coding sequence enabled the vg to be packaged successfully into the scAAV-mCMV-ΔhIDUA^{op} vector.

Codon-optimization and Miniaturization Enhance the Expression and Secretion of rIDUA: A side-by-side comparison of the therapeutic potential of the three AAV-hIDUA vector constructs, independent of vector transduction, was made after in vitro transfection. While all three constructs mediated above normal levels of IDUA activity in both the cell lysates (FIG. 3A) and media (FIG. 3B), ptrs-mCMV-hIDUA^{op} resulted in higher IDUA activity in both cell lysate (FIG. 3A) and media (FIG. 3B), by approximately 25% in total IDUA activity (FIG. 3C), compared to ptrs-mCMV-hIDUA. Surprisingly, the ptrs-mCMV-ΔhIDUA^{op} led to further a significant increase in IDUA activity in both cell lysate (3.1 fold) (FIG. 3A) and in media (11.1 fold) (FIG. 3B), with total 8.0 fold increase (FIG. 3C) over ptrs-mCMV-hIDUA^{op}. Importantly, these improvements were from transfected plasmids, all of which enter the cell as dsDNA. The enhanced transduction capability of the scAAV vector is expected to multiply this differential benefit. These data demonstrate that the codon-optimization and especially the miniaturization of hIDUA cDNA significantly enhanced the expression and secretion of the recombinant enzyme, strongly supporting the therapeutic potential of the scAAV9-mCMV-ΔhIDUA gene therapy product for treating MPS I.

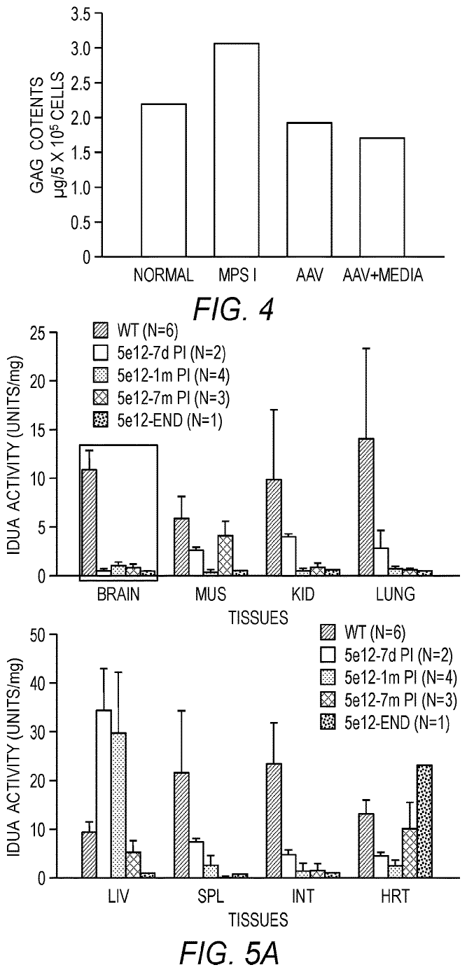

scAAV-ΔhIDUA^{op} Gene Delivery Mediates the Correction and Cross-correction of GAG Storage in vitro in MPS I Cells: Human MPS I skin fibroblasts (GM01257) were transfected in duplicate with ptrs-mCMV-ΔhIDUA^{op} plasmid to demonstrate the functionality of the scAAV-mediated recombinant ΔhIDUA (plasmid transfection was used because the AAV9 serotype in which the first batch of scAAV-ΔhIDUA^{op} vector was produced did not effectively transduce fibroblasts). At 48 hours post-transfection, the media from transfected cells were applied to non-treated GM01257 cells for 48 hours incubation. At 48 hours post transfection or media incubation, cell lysates were assayed in duplicate for GAG content. The results showed reduction of GAG content to normal levels in MPS I cells transduced with the vector construct or incubated with the media from ΔhIDUA^{op}-transfected GM01257 cells (FIG. 4). The data demonstrated that the AAV-mediated rΔIDUA is fully functional and led to the correction and cross-correction of GAG storage in MPS I cells. The data further supported the therapeutic potential of the proposed scAAV9-mCMV-ΔhIDUA^{op} vector for treating MPS I in humans.

### Example 2

Pre-Clinical Studies of rAAV-mCMV-hIDUA Vector without Codon-Optimization

Therapeutic impact of systemic rAAV9-hIDUA gene delivery for treating MPS I in mice: The rAAV9-mCMV-hIDUA vector has been tested in pre-clinical studies in MPS I mice via systemic delivery (Table 2). MPS I mice at age 1 month were treated with an IV injection of rAAV9-mCMV-hIDUA vector at 2×10¹² vg/kg (n=11) or 5×10¹² vg/kg (n=17), or at age 6 months with 1×10¹³ vg/kg (n=29). Mice were tested for behavior performance in a hidden task in Morris water maze at age 8 months (n=8-29/group). Necropsies were performed for tissue analyses at 7 days, 1 month, 7 months, or 12 months post vector injection, or humane endpoint. Subsets of mice per cohort were observed for longevity. Controls were non-treated MPS I and wild-type littermates.

TABLE 2
Study design: systemic rAAV9-mCMV-hIDUA gene delivery in MPS I mice

| | | | Number of animals (n) | | | | | | |
|---|---|---|---|---|---|---|---|---|---|
| | | Vector | | | Necropsy | | | | |
| | AOJ | dose | | Behavior | 7 d | 1 m | 7 m | End- | |
| Cohort | (m) | (vg/kg) | Total | test | pi | pi | pi | point | Longevity |
| 1. MPS I | 1 | 2 × 10¹² | 11 | 11* | — | — | — | 1 | 11 |
| 2. MPS I | 1 | 5 × 10¹² | 17 | 8* | 2 | 4 | 4 | 1 | 6 |
| 3. MPS I | 6 | 1 × 10¹³ | 29 | 29* | — | — | — | 9 | 29 |
| 4. MPS I | — | NT | 40 | 21* | — | 3 | 4 | — | 23 |
| 5. WT | — | NT | 27 | 21* | — | 3 | 3 | — | 21 |

AOJ: age of injection; NT: non-treated; pi: post vector injection; *behavior test at age 7 m.

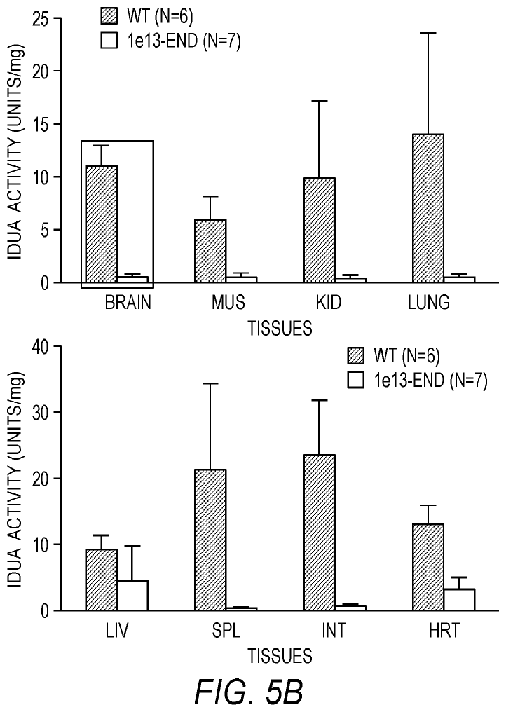

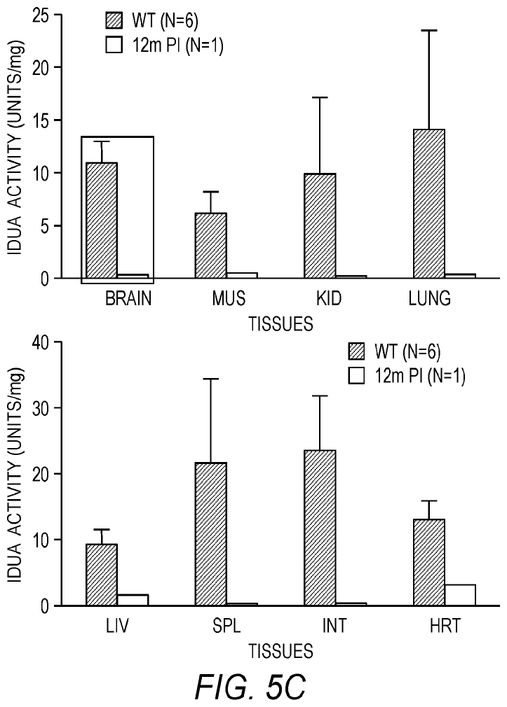

Rapid and Persistent Global Restoration of Functional IDUA Protein in the CNS, PNS and Somatic Tissues: The levels and persistence of transgene expression were assessed. Tissues from the mice in Table 2 were assayed for IDUA activity at 7d pi, 1 m pi, 7 m pi, or humane endpoint. An IV injection of 5×10¹² vg/kg rAAV9-mCMV-hIDUA vector resulted in rapid and persistent restoration of IDUA activity to supranormal levels in the liver, close to wild-type levels in the heart, sub-normal levels in lung, skeletal muscle, spleen and intestine, and up to 14% of wild-type levels in the brain at all time points (FIG. 5A). There was a decrease over time between 1 month post vector injection, 8 months, and endpoint. At endpoint, IDUA activity was detected at subnormal levels in the majority of the tested tissues and about 5% of wild-type levels in the brain in MPS I mice treated at age 6 months with 1×10¹³ vg/kg of the vector (FIG. 5B). The results also showed low IDUA activity in MPS I mice, who received an IV injection of 2×10¹² vg/kg rAAV9-mCMV-hIDUA at age 1 month. The mice had IDUA activity <2% of wild-type level in the brain, <25% of wild-type level in the liver and heart, and <5% of wild-type level in 5 other peripheral tissues when assayed at 12 months post vector injection (FIG. 5C). The data demonstrated that the vector treatment led to the quick and persistent restoration of functional IDUA in the CNS and broad peripheral tissues. Importantly, the AAV-mediated IDUA activity persisted to the endpoints in all tissues (FIGS. 5A-5C). The rIDUA expression is dose dependent and 2×10¹² vg/kg may not mediate sufficient rIDUA expression for functional neurological benefit. Notably, there was no detectable IDUA activity in tissues from non-treated MPS I mice.

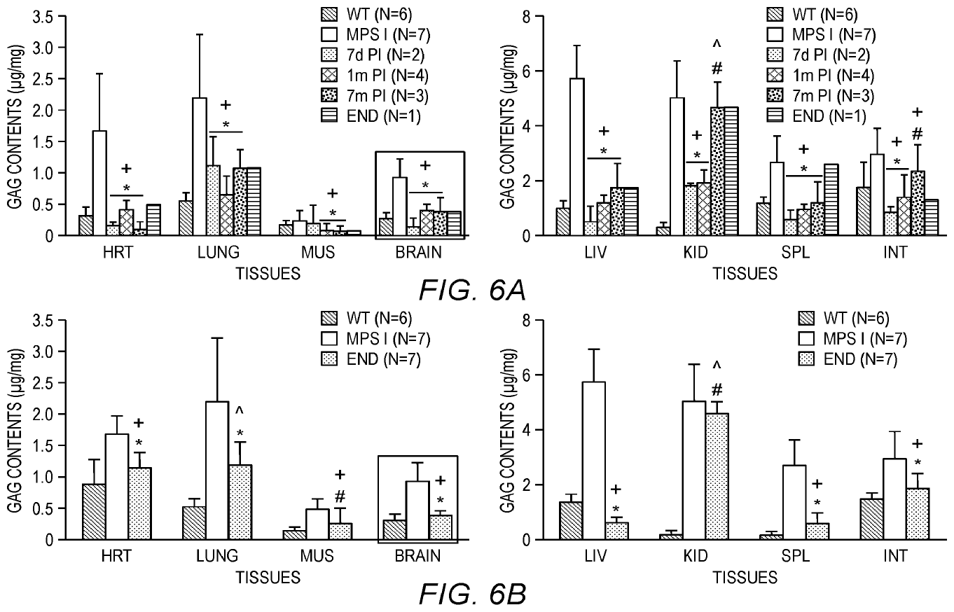

Clearance of Lysosomal GAG Storage Pathology and Astrocytosis: Tissues were assayed for GAG contents at different time points post vector injection to further assess the functionality of AAV9-mediated rIDUA. The results showed that an IV injection of 5×10¹² vg/kg rAAV9-mCMV-hIDUA vector at age 1 month or 1×10¹³ vg/kg at age 6 months resulted in significant reduction of GAG content to normal levels in all vector treated MPS I mice for the brain and the majority of the tested peripheral tissues, except the kidney (FIGS. 6A-6B). The clearance of tissue GAGs was rapid and persisted to the endpoint, correlating to the tissue rIDUA levels in FIGS. 5A-5C. The data demonstrated that the AAV-mediated rIDUA is functional and led to the correction of lysosomal storage pathology at effective doses.

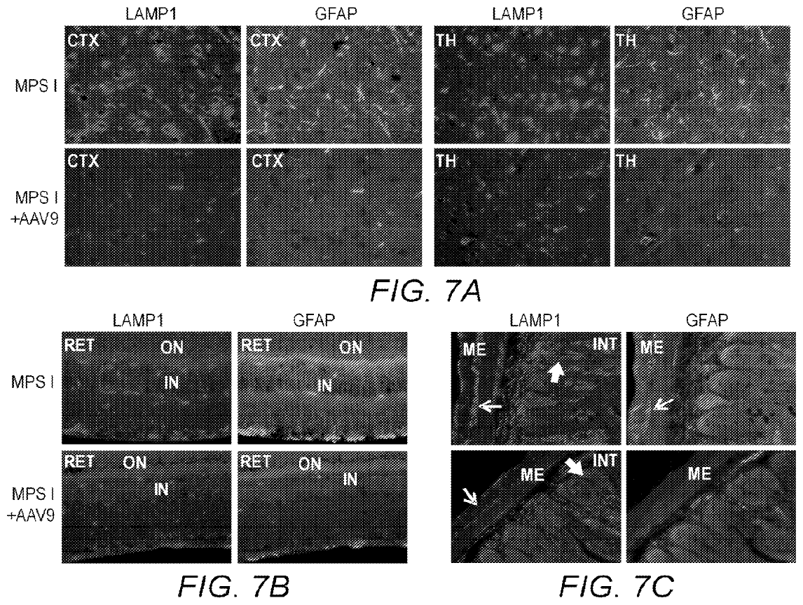

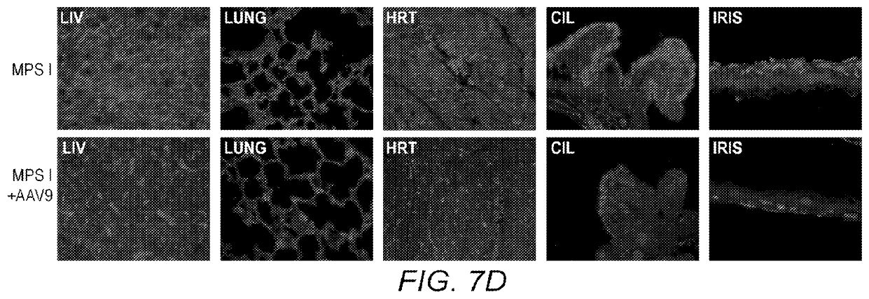

Further, immunofluorescence (IF) staining showed clearance of the LAMP1 lysosomal marker throughout the brain (FIG. 7A), intestinal nerves (FIG. 7B) and eye (FIG. 7C), and the majority of the tested somatic tissues (FIG. 7D). The data further supported that rAAV9-mediated rIDUA is functional and led to rapid and complete clearance of GAG storage in the CNS, peripheral nervous system (PNS) and optical nervous system (ONS), as well as broad peripheral tissues. IF staining also showed the clearance of GFAP-positive signals in the brain, intestine and retina (FIGS. 7A-7C), indicating amelioration of astrocytosis and neuroinflammation in the CNS, PNS and ONS.

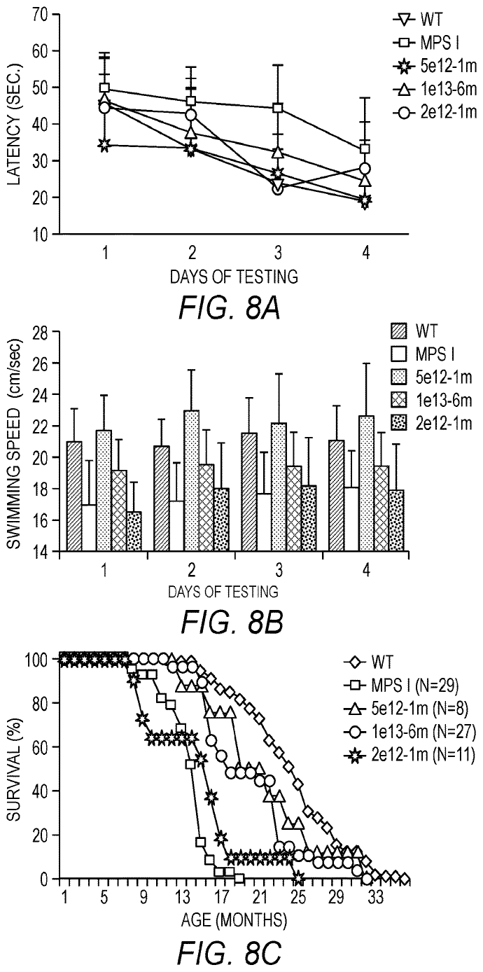

Functional Benefits: Correction of Behavior Deficits and Extension of Survival: Vector-treated MPS I mice and controls were tested for cognitive ability and motor function in a hidden task in the Morris water maze at 8 months to assess the functional impacts of systemic rAAV9-hIDUA gene delivery. The results showed normalized latency to find a hidden platform (FIG. 8A) and swimming ability (FIG. 8B) in MPS I mice that received an IV injection of 5×10¹² vg/kg rAAV9-mCMV-hIDUA at age 1 month, indicating the correction of cognitive and motor functions. However, MPS I mice treated at age 1 month with the vector at 2×10¹² vg/kg did not show detectable improvement in either the latency to the target (FIG. 8A) or the swimming speed (FIG. 8B), indicating that 2×10¹² vg/kg is below the minimal effective dose. Furthermore, improved, though not normalized, swimming ability (FIG. 8B) and latency to the find the hidden platform (FIG. 8A) was observed in MPS I mice that were treated at age 6 months with 1×10¹³ vg/kg vector. When the test results were analyzed for the 8 months cohort, only 2 months post vector injection (FIGS. 8A-8B), it was hypothesized that the full scale of functional benefits may require more time when treated at very advance disease stages. Notably, the majority of non-treated MPS I mice >8 months of age are not testable in water maze due to disease severity. The data demonstrated that a single IV injection of rAAV9-mCMV-hIDUA at an effective dose does not only prevent but may also reverse the cognitive and motor function deficits to a certain extent in MPS I mice.

A subset of each cohort was observed for longevity to further assess the functional benefits of the vector treatments. Significant extension of survival was observed in MPS I mice treated at age 1 month with 5×10¹² vg/kg rAAV9-mCMV-hIDUA or at age 6 months with 1×10¹³ vg/kg vector compared to non-treated MPS I mice (p<0.05) (FIG. 8C). Notably, the majority of these vector treated MPS I mice survived to within the normal lifespan range (FIG. 8C). Given that neurological manifestation is one of the major causes of premature death in MPS I, the significantly extended survival of treated MPS I mice further supports the functional therapeutic benefits of systemic rAAV9-mCMV-hIDUA gene delivery for treating MPS I.

However, vector treatment at 2×10¹² vg/kg did not show significant impact on the survival of MPS I mice, further supporting that vector treatment at 2×10¹² vg/kg is below the minimal effective dose, because it did not mediate sufficient rIDUA expression for the functional correction of MPS I.

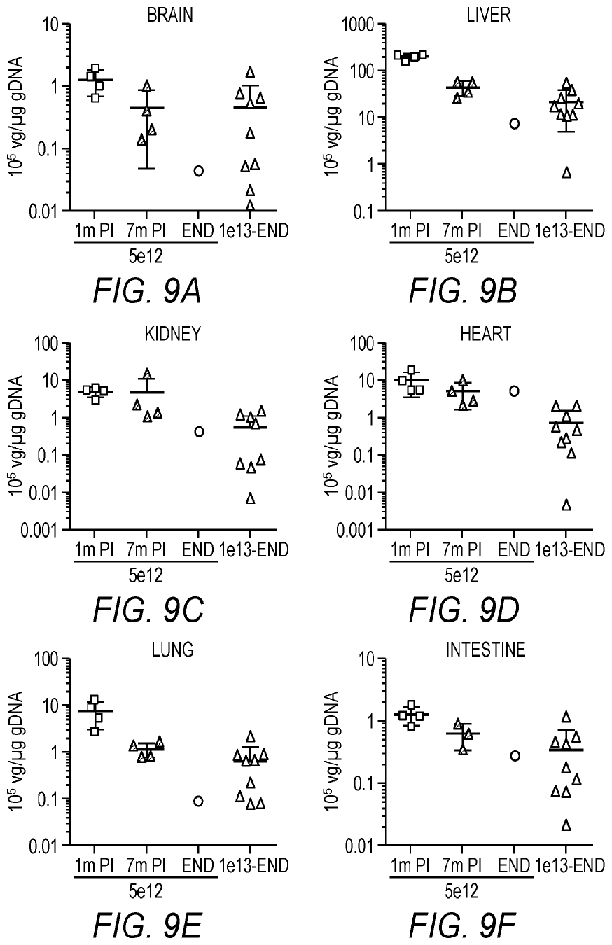

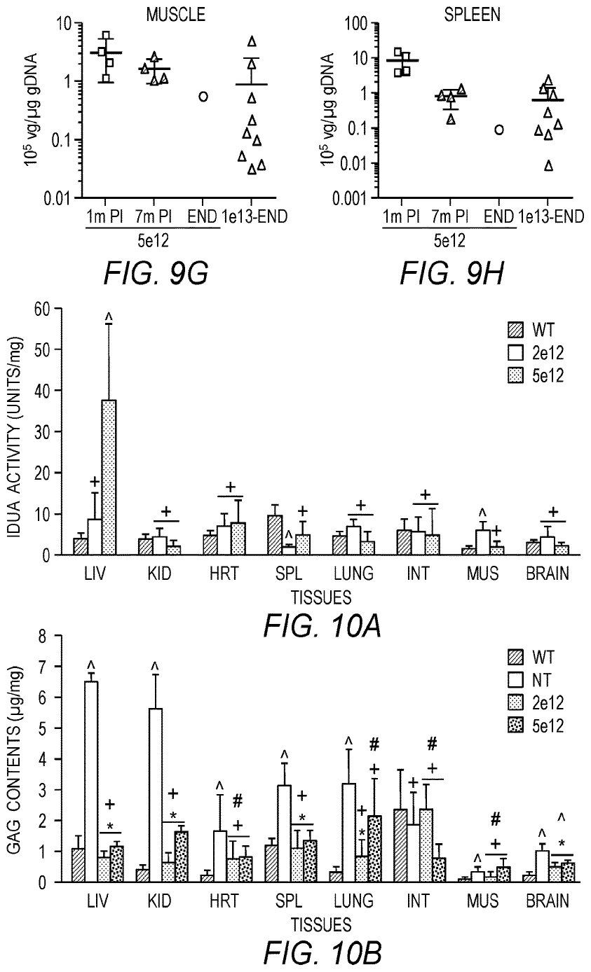

Differential Biodistribution of rAAV9-mCMV-hIDUA Vector Genome: Total DNA isolated from tissues was assayed by qPCR to determine the biodistribution of the systemically delivered rAAV9-mCMV-hIDUA and to quantify scAAV9-hIDUA vector genome copy numbers in tissues at different time points post vector injection (n≥4/group). The results showed differential biodistribution of the vector DNA among different tissues with the highest concentration detected in the liver, followed by heart, skeletal muscle, lung, intestine, kidney, spleen, and brain (FIGS. 9A-9H). Copies of the vector genome persisted in tissues, though decreased in some tissues over time between age 2 months and 8 months.

In Summary: Three gene replacement therapy vectors for treating MPS I, rAAV-mCMV-hIDUA, rAAV-mCMV-hIDUA^{op}, and scAAV-mCMV-ΔhIDUA^{op}, were developed. The data demonstrated that codon-optimization enhanced both the expression and secretion of rIDUA. Importantly, the deletion of 15 bp from the 3′-end non-conserved sequence enabled the packaging of scAAV vector, scAAV-mCMV-ΔhIDUA^{op}, which led to a further 11-fold improvement in rIDUA expression and secretion. Administration of the vector, rAAV-mCMV-hIDUA, was tested in MPS I mice. The preclinical study results reinforced the view that systemic delivery of the trans-BBB-neurotropic AAV9 gene therapy vector can prevent and reverse the global neuropathy and broad somatic manifestations of the disease. The minimal effective dose of rAAV-mCMV-hIDUA was determined to be 5×10¹² vg/kg. It is believed that scAAV-mCMV-ΔhIDUA^{op} will offer much greater therapeutic benefits, given the demonstrated higher transduction efficiency of scAAV vector, over the single-stranded rAAV-mCMV-hIDUA vector. Moreover, it is believed that much less scAAV-mCMV-ΔhIDUA^{op} vector will be needed for maximal efficacy, which will reduce the challenge in scale-up manufacture and potential toxicity.

### Example 3

Pre-Clinical Studies of scAAV-mCMV-ΔhIDUA^{op} Vector in MPS I Mouse Model

Preclinical studies were initiated to test the therapeutic potential of systemic delivery of scAAV9-mCMV-ΔhIDUA^{op} vector for treating MPS IIIC in mice. MPS I mice were treated at age 1 month with an IV injection of scAAV9-mCMV-ΔhIDUA^{op} vector at 2×10¹² vg/kg (n=12), 5×10¹² vg/kg (n=20), or 1×10¹³ vg/kg (n=16). For tissue analyses, necropsies were performed at 1 month post injection (pi)(n=4), and will be performed at 7 months pi (n=4) and humane endpoint. Mice will be tested for behavior performance in a hidden task in Morris water maze at age 8 months (n≥8/group). Subsets of mice per cohort will be observed for longevity. Controls were non-treated MPS I and wt littermates. The following are the results obtained so far.

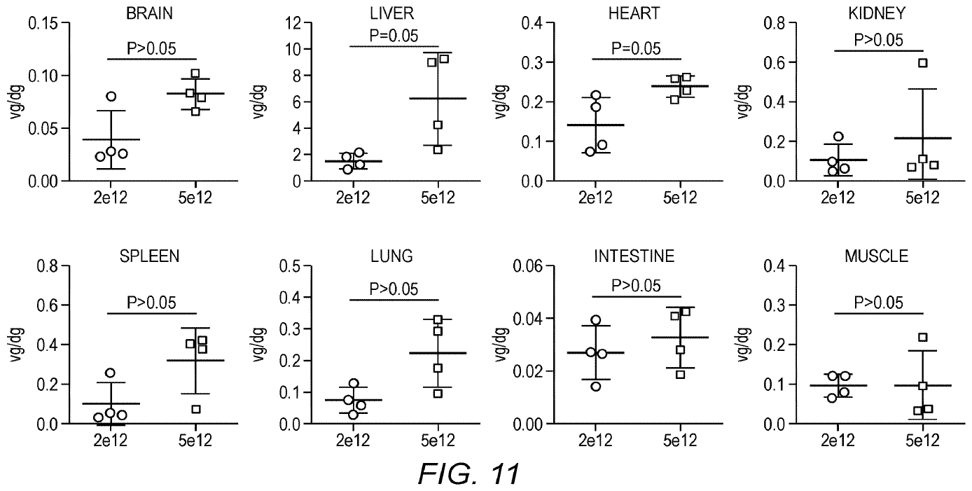

Rapid restoration of functional IDUA protein in the CNS and peripheral tissues: To assess the levels and persistence of transgene expression, tissues were assayed for IDUA activity at 1 month pi. An IV injection of 2×10¹² vg/kg scAAV9-mCMV-ΔhIDUA^{op} vector resulted in rapid restoration of IDUA activity to normal or close to normal levels in the liver, heart, kidney, lung, intestine and supranormal levels in brain and muscle, and sub-normal levels in spleen (FIG. 1a). The vector treatment at 5×10¹² vg/kg led to the restoration of IDUA activity to supranomal levels in the liver, to or close to normal levels in the brain, heart, kidney, lung, intestine, spleen and muscle (FIG. 10A). Notably, there was no detectable IDUA activity in tissues from non-treated MPS II mice. These data demonstrate that an IV scAAV9-mCMV-ΔhIDUA^{op} vector delivery mediated efficient expression of functional rIDUA protein leading to the quick restoration of IDUA activity in the CNS and broad peripheral tissues.

Clearance of GAG contents in the CNS and peripheral tissues: Tissues were also assayed for GAG contents at 1 month pi, to further assess the functionality of AAV9-mediated rIDUA. The results showed significant reduction of GAG contents in MPS I mice that an IV injection of scAAV9-mCMV-ΔhIDUA^{op} at both 2×10¹² vg/kg and 5×10¹² vg/kg resulted in significant reduction of GAG content to normal levels in the brain and the majority of the tested peripheral tissues, including liver, heart, kidney and spleen, in all vector treated MPS I mice (FIG. 10B). The GAG reduction was seen to be inconsistent in lung, intestine and muscle, due to the individual variation (FIG. 10B). These data further demonstrate that the AAV-mediated rIDUA is functional, leading to the quick tissue GAG clearance, supporting the correction of lysosomal storage pathology in the CNS and peripheral organs.

Differential biodistribution of rAAV9-mCMV-hIDUA vector genome: Total DNA isolated from tissues was assayed by qPCR to quantify scAAV9-hIDUA vector genome copy numbers in tissues at different time points post vector injection (n=4/group), to determine the biodistribution of the systemically delivered rAAV9-mCMV-hIDUA. The results showed differential biodistribution of the vector DNA among different tissues, with the highest concentration detected in the liver, followed by spleen, heart, lung, intestine, kidney, skeletal muscle, and brain (FIG. 11). The tissue vector copy levels were largely dose-responsive in the majority of tested tissues, including brain, liver, heart, spleen, and lung (FIG. 11).

In summary, preclinical studies demonstrate that the scAAV-mCMV-ΔhIDUA^{op} vector via an IV injection mediated the efficient expression of functional rIDUA protein, leading to the correction of lysosomal storage pathology in an MPS I mouse model. These can be achieved at a dose as low as 2×10¹² vg/kg, strongly supporting the therapeutic potential of scAAV-mCMV-ΔhIDUA^{op} for treating MPS I via a systemic delivery.

All publications, patents, and patent applications are herein incorporated by reference to the same extent as if each individual publication, patent, or patent application was specifically and individually indicated to be incorporated by reference.

Although the foregoing invention has been described in some detail by way of illustration and example for purposes of clarity of understanding, it will be obvious that certain changes and modifications may be practiced within the scope of the list of the foregoing embodiments and the appended claims.

That which is claimed is:

1. A recombinant nucleic acid comprising a nucleotide sequence encoding human alpha-L-iduronidase (IDUA), wherein the nucleotide sequence has been codon-optimized for expression in human cells and wherein the nucleotide sequence is at least 90% identical to SEQ ID NO:1.

2. A recombinant nucleic acid comprising a nucleotide sequence encoding human alpha-L-iduronidase (IDUA), wherein the nucleotide sequence has been truncated by removing 15 nucleotides of the non-coding sequence from near the 3′ end and wherein the nucleotide sequence is at least 90% identical to SEQ ID NO:2.

3. The recombinant nucleic acid of claim 1, wherein the nucleotide sequence has been truncated by removing 15 nucleotides of the non-coding sequence from near the 3′ end and wherein the nucleotide sequence is at least 90% identical to SEQ ID NO:3.

4. The recombinant nucleic acid of claim 1 comprising the nucleotide sequence of SEQ ID NO:1.

5. The recombinant nucleic acid of claim 2 comprising the nucleotide sequence of SEQ ID NO:2.

6. The recombinant nucleic acid of claim 3 comprising the nucleotide sequence of SEQ ID NO:3.

7. An adeno-associated virus (AAV) vector genome comprising the nucleic acid of any one of claims 1-6.

8. The AAV vector genome of claim 7, wherein the nucleic acid is operably linked to a promoter.

9. The AAV vector genome of claim 8, wherein the promoter is 250 base pairs or less in length.

10. The AAV vector genome of claim 9, wherein the promoter is a mCMV promoter.

11. The AAV vector genome of claim 10, wherein the mCMV promoter sequence is identical to SEQ ID NO:7.

12. The AAV vector genome of any one of claims 7-11, wherein the nucleic acid is operably linked to a polyadenylation signal.

13. The AAV vector genome of claim 12, wherein and the polyadenylation signal is less than 25 base pairs in length.

14. The AAV vector genome of claim 13, wherein the polyadenylation signal is a sNRP-1 polyadenylation signal.

15. The AAV vector genome of claim 14, wherein the sNRP-1 polyadenylation signal is identical to SEQ ID NO:8.

16. The AAV vector genome of any one of claims 7-15, wherein the AAV vector genome is self-complementary.

17. A cell in vitro comprising the AAV vector genome of any one of claims 7-16.

18. The cell of claim 17, wherein the AAV vector genome is stably incorporated into the cell genome.

19. An AAV particle comprising the AAV vector genome of any one of claims 7-16.

20. A method of producing a recombinant AAV particle, the method comprising:
providing a cell in vitro with AAV Cap and AAV Rep coding sequences, the AAV vector genome of any one of claims 7-16, and helper functions for generating a productive AAV infection; and
allowing assembly of the recombinant AAV particle encapsidating the AAV vector genome.

21. An AAV particle produced by the method of claim 20.

22. The AAV particle of any one of claim 19 or 21, wherein the AAV particle is an AAV2, AAV8, or AAV9 particle.

23. The AAV particle of any one of claim 19 or 21, wherein the AAV particle is a chimeric AAV8/AAV9 particle.

24. A pharmaceutical formulation comprising the AAV particle of any one of claim 19 or 21-23 in a pharmaceutically acceptable carrier.

25. A method of delivering IDUA to a subject, comprising administering to the subject an effective amount of the AAV particle of any one of claim 19 or 21-23, thereby delivering IDUA to the subject.

26. A method of treating or delaying the onset of mucopolysaccharidosis I (MPS I) in a subject in need thereof, comprising administering to the subject a therapeutically effective amount of the AAV particle of any one of claim 19 or 21-23, thereby treating or delaying the onset of MPS I in the subject.

27. A method of delivering IDUA to a cell in vitro or ex vivo, comprising contacting the cell with an effective amount of the AAV particle of any one of claim 19 or 21-23, thereby delivering IDUA to the cell.

28. The method of claim 27, further comprising transplanting the cell to a subject in need thereof.

29. The method of any one of claims 25-28, wherein the AAV particle is administered to the subject by injection.

30. The method of any one of claims 25-28, wherein the AAV particle is administered to the subject orally or topically.

31. The method of any one of claims 25-30, wherein the subject is a human subject.

32. The method of any one of claims 25-31, wherein the subject has been diagnosed with MPS I.
